SEQUENCE LISTING (9 sequences)

SEQ ID NO: 1 - protein, 447 aa, Homo sapiens
MSLWLGAPVPDIPPDSAVELWKPGAQDASSQAQGGSSCILREEARMPHSAGGTAGVGLEAAEPTALLTRAEPPSEPTEIRPQKRKKGPAPKMLGNELCSVCGDKASGFHYNVLSCEGCKGFFRRSVIKGAHYICHSGGHCPMDTYMRRKCQECRLRKCRQAGMREECVLSEEQIRLKKLKRQEEEQAHATSLPPRRSSPPQILPQLSPEQLGMIEKLVAAQQQCNRRSFSDRLRVTPWPMAPDPHSREARQQRFAHFTELAIVSVQEIVDFAKQLPGFLQLSREDQIALLKTSAIEVMLLETSRRYNPGSESITFLKDFSYNREDFAKAGLQVEFINPIFEFSRAMNELQLNDAEFALLIAISIFSADRPNVQDQLQVERLQHTYVEALHAYVSIHHPHDRLMFPRMLMKLVSLRTLSSVHSEQVFALRLQDKKLPPLLSEIWDVHE

SEQ ID NO: 2 - DNA, 1344 bp, Homo sapiens
atgtccttgtggctgggggcccctgtgcctgacattcctcctgactctgcggtggagctgtggaagccaggcgcacaggatgcaagcagccaggcccagggaggcagcagctgcatcctcagagaggaagccaggatgccccactctgctgggggtactgcaggggtggggctggaggctgcagagcccacagccctgctcaccagggcagagcccccttcagaacccacagagatccgtccacaaaagcggaaaaaggggccagcccccaaaatgctggggaacgagctatgcagcgtgtgtggggacaaggcctcgggcttccactacaatgttctgagctgcgagggctgcaagggattcttccgccgcagcgtcatcaagggagcgcactacatctgccacagtggcggccactgccccatggacacctacatgcgtcgcaagtgccaggagtgtcggcttcgcaaatgccgtcaggctggcatgcgggaggagtgtgtcctgtcagaagaacagatccgcctgaagaaactgaagcggcaagaggaggaacaggctcatgccacatccttgccccccaggcgttcctcacccccccaaatcctgccccagctcagcccggaacaactgggcatgatcgagaagctcgtcgctgcccagcaacagtgtaaccggcgctccttttctgaccggcttcgagtcacgccttggcccatggcaccagatccccatagccgggaggcccgtcagcagcgctttgcccacttcactgagctggccatcgtctctgtgcaggagatagttgactttgctaaacagctacccggcttcctgcagctcagccgggaggaccagattgccctgctgaagacctctgcgatcgaggtgatgcttctggagacatctcggaggtacaaccctgggagtgagagtatcaccttcctcaaggatttcagttataaccgggaagactttgccaaagcagggctgcaagtggaattcatcaaccccatcttcgagttctccagggccatgaatgagctgcaactcaatgatgccgagtttgccttgctcattgctatcagcatcttctctgcagaccggcccaacgtgcaggaccagctccaggtggagaggctgcagcacacatatgtggaagccctgcatgcctacgtctccatccaccatccccatgaccgactgatgttcccacggatgctaatgaaactggtgagcctccggaccctgagcagcgtccactcagagcaagtgtttgcactgcgtctgcaggacaaaaagctcccaccgctgctctctgagatctgggatgtgcacgaatga

SEQ ID NO: 3 - protein, 1263 aa, Homo sapiens
MLVKPLPDSEEEGHDNQEAHQKYETMQCFAVSQPKSIKEEGEDLQSCLICVARRVPMKERPVLPSSESFTTRQDLQGKITSLDTSTMRAAMKPGWEDLVRRCIQKFHAQHEGESVSYAKRHHHEVLRQGLAFSQIYRFSLSDGTLVAAQTKSKLIRSQTTNEPQLVISLHMLHREQNVCVMNPDLTGQTMGKPLNPISSNSPAHQALCSGNPGQDMTLSSNINFPINGPKEQMGMPMGRFGGSGGMNHVSGMQATTPQGSNYALKMNSPSQSSPGMNPGQPTSMLSPRHRMSPGVAGSPRIPPSQFSPAGSLHSPVGVCSSTGNSHSYTNSSLNALQALSEGHGVSLGSSLASPDLKMGNLQNSPVNMNPPPLSKMGSLDSKDCFGLYGEPSEGTTGQAESSCHPGEQKETNDPNLPPAVSSERADGQSRLHDSKGQTKLLQLLTTKSDQMEPSPLASSLSDTNKDSTGSLPGSGSTHGTSLKEKHKILHRLLQDSSSPVDLAKLTAEATGKDLSQESSSTAPGSEVTIKQEPVSPKKKENALLRYLLDKDDTKDIGLPEITPKLERLDSKTDPASNTKLIAMKTEKEEMSFEPGDQPGSELDNLEEILDDLQNSQLPQLFPDTRPGAPAGSVDKQAIINDLMQLTAENSPVTPVGAQKTALRISQSTFNNPRPGQLGRLLPNQNLPLDITLQSPTGAGPFPPIRNSSPYSVIPQPGMMGNQGMIGNQGNLGNSSTGMIGNSASRPTMPSGEWAPQSSAVRVTCAATTSAMNRPVQGGMIRNPAASIPMRPSSQPGQRQTLQSQVMNIGPSELEMNMGGPQYSQQQAPPNQTAPWPESILPIDQASFASQNRQPFGSSPDDLLCPHPAAESPSDEGALLDQLYLALRNFDGLEEIDRALGIPELVSQSQAVDPEQFSSQDSNIMLEQKAPVFPQQYASQAQMAQGSYSPMQDPNFHTMGQRPSYATLRMQPRPGLRPTGLVQNQPNQLRLQLQHRLQAQQNRQPLMNQISNVSNVNLTLRPGVPTQAPINAQMLAQRQREILNQHLRQRQMHQQQQVQQRTLMMRGQGLNMTPSMVAPSGMPATMSNPRIPQANAQQFPFPPNYGISQQPDPGFTGATTPQSPLMSPRMAHTQSPMMQQSQANPAYQAPSDINGWAQGNMGGNSMFSQQSPPHFGQQANTSMYSNNMNINVSMATNTGGMSSMNQMTGQISMTSVTSVPTSGLSSMGPEQVNDPALRGGNLFPNQLPGMDMIKQEGDTTRKYC

SEQ ID NO: 4 - DNA, 6158 bp, Homo sapiens
ggcggccgcagcctcggctacagcttcggcggcgaaggtcagcgccgacggcagccggcacctgacggcgtgaccgacccgagccgatttctcttggatttggctacacacttatagatcttctgcactgtttacaggcacagttgctgatatgtgttcaagatgagtgggatgggagaaaatacctctgacccctccagggcagagacaagaaagcgcaaggaatgtcctgaccaacttggacccagccccaaaaggaacactgaaaaacgtaatcgtgaacaggaaaataaatatatagaagaacttgcagagttgatttttgcaaattttaatgatatagacaactttaacttcaaacctgacaaatgtgcaatcttaaaagaaactgtgaagcaaattcgtcagatcaaagaacaagagaaagcagcagctgccaacatagatgaagtgcagaagtcagatgtatcctctacagggcagggtgtcatcgacaaggatgcgctggggcctatgatgcttgaggcccttgatgggttcttctttgtagtgaacctggaaggcaacgttgtgtttgtgtcagagaatgtgacacagtatctaaggtataaccaagaagagctgatgaacaaaagtgtatatagcatcttgcatgttggggaccacacggaatttgtcaaaaacctgctgccaaagtctataggtaaatgggggatcttggtctggcgaacctccgaggcggaacagccataccttcaattgtcggatgctggtaaaacctttacctgattcagaagaggagggtcatgataaccaggaagctcatcagaaatatgaaactatgcagtgcttcgctgtctctcaaccaaagtccatcaaagaagaaggagaagatttgcagtcctgcttgatttgcgtggcaagaagagttcccatgaaggaaagaccagttcttccctcatcagaaagttttactactcgccaggatctccaaggcaagatcacgtctctggataccagcaccatgagagcagccatgaaaccaggctgggaggacctggtaagaaggtgtattcagaagttccatgcgcagcatgaaggagaatctgtgtcctatgctaagaggcatcatcatgaagtactgagacaaggattggcattcagtcaaatctatcgtttttccttgtctgatggcactcttgttgctgcacaaacgaagagcaaactcatccgttctcagactactaatgaacctcaacttgtaatatctttacatatgcttcacagagagcagaatgtgtgtgtgatgaatccggatctgactggacaaacgatggggaagccactgaatccaattagctctaacagccctgcccatcaggccctgtgcagtgggaacccaggtcaggacatgaccctcagtagcaatataaattttcccataaatggcccaaaggaacaaatgggcatgcccatgggcaggtttggtggttctgggggaatgaaccatgtgtcaggcatgcaagcaaccactcctcagggtagtaactatgcactcaaaatgaacagcccctcacaaagcagccctggcatgaatccaggacagcccacctccatgctttcaccaaggcatcgcatgagccctggagtggctggcagccctcgaatcccacccagtcagttttcccctgcaggaagcttgcattcccctgtgggagtttgcagcagcacaggaaatagccatagttataccaacagctccctcaatgcacttcaggccctcagcgaggggcacggggtctcattagggtcatcgttggcttcaccagacctaaaaatgggcaatttgcaaaactccccagttaatatgaatcctcccccactcagcaagatgggaagcttggactcaaaagactgttttggactatatggggagccctctgaaggtacaactggacaagcagagagcagctgccatcctggagagcaaaaggaaacaaatgaccccaacctgcccccggccgtgagcagtgagagagctgacgggcagagcagactgcatgacagcaaagggcagaccaaactcctgcagctgctgaccaccaaatctgatcagatggagccctcgcccttagccagctctttgtcggatacaaacaaagactccacaggtagcttgcctggttctgggtctacacatggaacctcgctcaaggagaagcataaaattttgcacagactcttgcaggacagcagttcccctgtggacttggccaagttaacagcagaagccacaggcaaagacctgagccaggagtccagcagcacagctcctggatcagaagtgactattaaacaagagccggtgagccccaagaagaaagagaatgcactacttcgctatttgctagataaagatgatactaaagatattggtttaccagaaataacccccaaacttgagagactggacagtaagacagatcctgccagtaacacaaaattaatagcaatgaaaactgagaaggaggagatgagctttgagcctggtgaccagcctggcagtgagctggacaacttggaggagattttggatgatttgcagaatagtcaattaccacagcttttcccagacacgaggccaggcgcccctgctggatcagttgacaagcaagccatcatcaatgacctcatgcaactcacagctgaaaacagccctgtcacacctgttggagcccagaaaacagcactgcgaatttcacagagcacttttaataacccacgaccagggcaactgggcaggttattgccaaaccagaatttaccacttgacatcacattgcaaagcccaactggtgctggacctttcccaccaatcagaaacagtagtccctactcagtgatacctcagccaggaatgatgggtaatcaagggatgataggaaaccaaggaaatttagggaacagtagcacaggaatgattggtaacagtgcttctcggcctactatgccatctggagaatgggcaccgcagagttcggctgtgagagtcacctgtgctgctaccaccagtgccatgaaccggccagtccaaggaggtatgattcggaacccagcagccagcatccccatgaggcccagcagccagcctggccaaagacagacgcttcagtctcaggtcatgaatatagggccatctgaattagagatgaacatggggggacctcagtatagccaacaacaagctcctccaaatcagactgccccatggcctgaaagcatcctgcctatagaccaggcgtcttttgccagccaaaacaggcagccatttggcagttctccagatgacttgctatgtccacatcctgcagctgagtctccgagtgatgagggagctctcctggaccagctgtatctggccttgcggaattttgatggcctggaggagattgatagagccttaggaatacccgaactggtcagccagagccaagcagtagatccagaacagttctcaagtcaggattccaacatcatgctggagcagaaggcgcccgttttcccacagcagtatgcatctcaggcacaaatggcccagggtagctattctcccatgcaagatccaaactttcacaccatgggacagcggcctagttatgccacactccgtatgcagcccagaccgggcctcaggcccacgggcctagtgcagaaccagccaaatcaactaagacttcaacttcagcatcgcctccaagcacagcagaatcgccagccacttatgaatcaaatcagcaatgtttccaatgtgaacttgactctgaggcctggagtaccaacacaggcacctattaatgcacagatgctggcccagagacagagggaaatcctgaaccagcatcttcgacagagacaaatgcatcagcaacagcaagttcagcaacgaactttgatgatgagaggacaagggttgaatatgacaccaagcatggtggctcctagtggtatgccagcaactatgagcaaccctcggattccccaggcaaatgcacagcagtttccatttcctccaaactacggaataagtcagcaacctgatccaggctttactggggctacgactccccagagcccacttatgtcaccccgaatggcacatacacagagtcccatgatgcaacagtctcaggccaacccagcctatcaggccccctccgacataaatggatgggcgcaggggaacatgggcggaaacagcatgttttcccagcagtccccaccacactttgggcagcaagcaaacaccagcatgtacagtaacaacatgaacatcaatgtgtccatggcgaccaacacaggtggcatgagcagcatgaaccagatgacaggacagatcagcatgacctcagtgacctccgtgcctacgtcagggctgtcctccatgggtcccgagcaggttaatgatcctgctctgaggggaggcaacctgttcccaaaccagctgcctggaatggatatgattaagcaggagggagacacaacacggaaatattgctgacactgctgaagccagttgcttcttcagctgaccgggctcacttgctcaaaacacttccagtctggagagctgtgtctatttgtttcaacccaactgacctgccagccggttctgctagagcagacaggcctggccctggttcccagggtggcgtccactcggctgtggcaggaggagctgcctcttctcttgacagtctgaagctcgcatccagacagtcgctcagtctgttcactgcattcaccttagtgcaacttagatctctcctgcaaaagtaaatgttgacaggcaaatttcatacccatgtcagattgaatgtatttaaatgtatgtatttaaggagaaccatgctcttgttctgttcctgttcggttccagacactggtttcttgctttgttttccctggctaacagtctagtgcaaaagattaagattttatctgggggaaagaaaagaattttttaaaaaattaaactaaagatgttttaagctaaagcctgaatttgggatggaagcaggacagacaccgtggacagcgctgtatttacagacacacccagtgcgtgaagaccaacaaagtcacagtcgtatctctagaaagctctaaagaccatgttggaaagagtctccagttactgaacagatgaaaaggagcctgtgagagggctgttaacattagcaaatattttttccttgttttttctttgttaaaaccaaactggttcacctgaatcatgaattgagaagaaataattttcatttctaaattaagtcccttttagtttgatcagacagcttgaatcagcatctcttcttccctgtcagcctgactcttcccttcccctctctcattccccatactccctattttcattccttttttaaaaaataatataagctacagaaaccaggtaagccctttatttccttaaatgttttgccagccacttaccaattgctaagtattgaatttcagaaaaaaaaaatgcatttactggcaaggagaagagcaaagttaaggcttgataccaatcgagctaaggatacctgctttggaagcatgtttattctgttccccagcaactctggcctccaaaatgggagaaaacgccagtgtgtttaaattgatagcagatatcacgacagatttaacctctgccatgtgttttttattttgttttttagcagtgctgactaagccgaagttttgtaaggtacataaaatccaatttatatgtaaacaagcaataatttaagttgagaacttatgtgttttaattgtataatttttgtgaggtatacatattgtggaattgactcaaaaatgaggtacttcagtattaaattagatatcttcatagcaatgtctcctaaaggtgttttgtaaaggatatcaatgccttgattagacctaatttgtagacttaagactttttattttctaaaccttgtgattctgcttataagtcatttatctaatctatatgatatgcagccgctgtaggaaccaattcttgatttttatatgtttatattctttcttaatgaaccttagaaagactacatgttactaagcaggccacttttatggttgttttt

SEQ ID NO: 5 - protein, 460 aa, Homo sapiens
MSSPTTSSLDTPLPGNGPPQPGAPSSSPTVKEEGPEPWPGGPDPDVPGTDEASSACSTDWVIPDPEEEPERKRKKGPAPKMLGHELCRVCGDKASGFHYNVLSCEGCKGFFRRSVVRGGARRYACRGGGTCQMDAFMRRKCQQCRLRKCKEAGMREQCVLSEEQIRKKKIRKQQQESQSQSQSPVGPQGSSSSASGPGASPGGSEAGSQGSGEGEGVQLTAAQELMIQQLVAAQLQCNKRSFSDQPKVTPWPLGADPQSRDARQQRFAHFTELAIISVQEIVDFAKQVPGFLQLGREDQIALLKASTIEIMLLETARRYNHETECITFLKDFTYSKDDFHRAGLQVEFINPIFEFSRAMRRLGLDDAEYALLIAINIFSADRPNVQEPGRVEALQQPYVEALLSYTRIKRPQDQLRFPRMLMKLVSLRTLSSVHSEQVFALRLQDKKLPPLLSEIWDVHE

SEQ ID NO: 6 - DNA, 1383 bp, Homo sapiens
atgtcctctcctaccacgagttccctggatacccccctgcctggaaatggcccccctcagcctggcgccccttcttcttcacccactgtaaaggaggagggtccggagccgtggcccgggggtccggaccctgatgtcccaggcactgatgaggccagctcagcctgcagcacagactgggtcatcccagatcccgaagaggaaccagagcgcaagcgaaagaagggcccagccccgaagatgctgggccacgagctttgccgtgtctgtggggacaaggcctccggcttccactacaacgtgctcagctgcgaaggctgcaagggcttcttccggcgcagtgtggtccgtggtggggccaggcgctatgcctgccggggtggcggaacctgccagatggacgctttcatgcggcgcaagtgccagcagtgccggctgcgcaagtgcaaggaggcagggatgagggagcagtgcgtcctttctgaagaacagatccggaagaagaagattcggaaacagcagcaggagtcacagtcacagtcgcagtcacctgtggggccgcagggcagcagcagctcagcctctgggcctggggcttcccctggtggatctgaggcaggcagccagggctccggggaaggcgagggtgtccagctaacagcggctcaagaactaatgatccagcagttggtggcggcccaactgcagtgcaacaaacgctccttctccgaccagcccaaagtcacgccctggcccctgggcgcagacccccagtcccgagatgcccgccagcaacgctttgcccacttcacggagctggccatcatctcagtccaggagatcgtggacttcgctaagcaagtgcctggtttcctgcagctgggccgggaggaccagatcgccctcctgaaggcatccactatcgagatcatgctgctagagacagccaggcgctacaaccacgagacagagtgtatcaccttcttgaaggacttcacctacagcaaggacgacttccaccgtgcaggcctgcaggtggagttcatcaaccccatcttcgagttctcgcgggccatgcggcggctgggcctggacgacgctgagtacgccctgctcatcgccatcaacatcttctcggccgaccggcccaacgtgcaggagccgggccgcgtggaggcgttgcagcagccctacgtggaggcgctgctgtcctacacgcgcatcaagaggccgcaggaccagctgcgcttcccgcgcatgctcatgaagctggtgagcctgcgcacgctgagctctgtgcactcggagcaggtcttcgccttgcggctccaggacaagaagctgccgcctctgctgtcggagatctgggacgtccacgagtga

SEQ ID NO: 7 - DNA, 49 bp, Homo sapiens
ggggacaagtttgtacaaaaaagcaggctcgcttcgcaaatgccgtcag

SEQ ID NO: 8 - DNA, 52 bp, Homo sapiens
ggggaccactttgtacaagaaagctgggtccccttctcagtctgttccactt

SEQ ID NO: 9 - DNA, 66 bp, Rattus norwegicus
ccctttggtcactcaagttcaagtgatgatagaattcggatcctttggtcactcaagttcaagtga

(19) United States
(12) Patent Application Publication
(10) Pub. No.: US 2005/0261319 A1
(43) Pub. Date: Nov. 24, 2005

(54) Novel 2-amino-4-quinazolinones and 2-amino-4-oxoquinazolones as LXR nuclear receptor binding compounds with partial agonistic properties

(76) Inventors: Ulrich Deuschle, Bammental (DE); Ralph Loebbert, Heidelberg (DE); Beatrix Blume, Dossenheim (DE); Manfred Koegl, Eppelheim (DE); Claus Kremoser, Heidelberg (DE); Ingo Kober, Gaiberg (DE); Ulrike Bauer, Sandhausen (DE); Kristina Hermann, Lampertheim (DE); Michael Albers, Dossenheim (DE)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/076,163

(22) Filed: Mar. 9, 2005

Related U.S. Application Data
(63) Continuation-in-part of application No. PCT/EP03/07067, filed on Jul. 2, 2003.
(63) Continuation-in-part of application No. PCT/EP03/10036, filed on Sep. 10, 2003.

(30) Foreign Application Priority Data
Sep. 10, 2002 (EP) 02 020 255.2

(51) Int. Cl. A61K 31/517; C07D 43/02

(52) U.S. Cl. 514/266.21; 514/266.3; 544/284; 544/285

(57) ABSTRACT

The present invention relates to compounds according to the general formulas (I) and/or (Ia), which bind to the LXR receptors and act as agonists and antagonists of the LXR receptors. The invention further relates to the treatment of diseases and/or conditions through binding of said nuclear receptor by said compounds and the production of medicaments using said compounds.

### CROSS-REFERENCE TO RELATED APPLICATIONS

The subject application is a continuation-in-part of International Application PCT/EP2003/07067, filed Jul. 2, 2003; which claims priority to EP Application No. 02020255.2, filed Sep. 10, 2002. This application is also a continuation-in-part of International Application No. PCT/EP2003/10036, filed Sep. 10, 2003; which also claims priority to EP 02 020 255.2, filed Sep. 10, 2002; both of which are herewith incorporated by reference in their entireties.

The present invention relates to compounds according to the general formulas (I) and/or (Ia), which bind to the LXR receptors and act as agonists and antagonists of the LXR receptors. The invention further relates to the treatment of diseases and/or conditions through binding of said nuclear receptor by said compounds and the production of medicaments using said compounds.

### BACKGROUND OF THE INVENTION

Multicellular organisms are dependent on advanced mechanisms of information transfer between cells and body compartments. The information that is transmitted can be highly complex and can result in the alteration of genetic programs involved in cellular differentiation, proliferation, or reproduction. The signals, or hormones, are often simple molecules, such as peptides, fatty acid, or cholesterol derivatives.

Many of these signals produce their effects by ultimately changing the transcription of specific genes. One well-studied group of proteins that mediate a cells response to a variety of signals is the family of transcription factors known as nuclear receptors, hereinafter referred to often as “NR”. Members of this group include receptors for steroid hormones, vitamin D, ecdysone, cis and trans retinoic acid, thyroid hormone, bile acids, cholesterol-derivatives, fatty acids (and other peroxisomal proliferators), as well as so-called orphan receptors, proteins that are structurally similar to other members of this group, but for which no ligands are known (Escriva, H. et al., Ligand binding was acquired during evolution of nuclear receptors, PNAS, 94, 6803-6808, 1997). Orphan receptors may be indicative of unknown signalling pathways in the cell or may be nuclear receptors that function without ligand activation. The activation of transcription by some of these orphan receptors may occur in the absence of an exogenous ligand and/or through signal transduction pathways originating from the cell surface (Mangelsdorf, D. J. etal., The nuclear receptor superfamily: the second decade, Cell 83, 835-839,1995).

In general, three functional domains have been defined in NRs. An amino terminal domain is believed to have some regulatory function. A DNA-binding domain hereinafter referred to as “DBD” usually comprises two zinc finger elements and recognizes a specific Hormone Responsive Element hereinafter referred to as “HRE” within the promoters of responsive genes.

Specific amino acid residues in the “DBD” have been shown to confer DNA sequence binding specificity (Schena, M. & Yamamoto, K. R., Mammalian Glucocorticoid Receptor Derivatives Enhance Transcription in Yeast, Science, 241: 965-967,1988). A Ligand-binding-domain hereinafter referred to as “LBD” is at the carboxy-terminal region of known NRs. In the absence of hormone, the LBD of some but not all NRs appears to interfere with the interaction of the DBD with its HRE. Hormone binding seems to result in a conformational change in the NR and thus opens this interference (Brzozowski et al., Molecular basis of agonism and antagonism in the oestrogen receptor, Nature, 389,753-758, 1997; Wagner et al., A structural role for hormone in the thyroid hormone receptor, Nature, 378,690-697. 1995). A NR without the HBD constitutively activates transcription but at a low level.

Coactivators or transcriptional activators are proposed to bridge between sequence specific transcription factors and the basal transcription machinery and in addition to influence the chromatin structure of a target cell. Several proteins like SRC-1, ACTR, and Grip1 interact with NRs in a ligand enhanced manner (Heery et al., A signature motif in transcriptional coactivators mediates binding to nuclear receptors, Nature, 387,733-736 ; Heinzel et al., A complex containing N—CoR, mSin3 and histone deacetylase mediates transcriptional repression, Nature 387, 43-47,1997). Furthermore, the physical interaction with repressing re-ceptor-interacting proteins or corepressors has been demonstrated (Xu et al., Coactivator and Corepressor complexes in nuclear receptor function, Curr Opin Genet Dev, 9 (2), 140-147, 1999).

Nuclear receptor modulators like steroid hormones affect the growth and function of specific cells by binding to intracellular receptors and forming nuclear receptor-ligand complexes.

Nuclear receptor-hormone complexes then interact with a hormone response element (HRE) in the control region of specific genes and alter specific gene expression.

Liver X Receptor (LXR) is a prototypical type 2 nuclear receptor which activates genes upon binding to promoter region of target genes in a prototypical heterodimeric fashion with Retinoid X Receptor (hereinafter RXR, Forman et al., Cell, 81, 687-93, 1995). The term “LXR” (Liver X Receptor) includes all subtypes of this receptor. Specifically LXR includes LXRa (also known as LXRalpha, RLD-1 and NR1H3) and LXRb (also known as LXRbeta, NER, NER1, UR, OR-1, R1P15 and NH1H2) and ligands of LXR should be understood to include ligands of LXRa or LXRb. LXR is a prototypical type 2 nuclear receptor which activates genes upon binding to promoter region of target genes in a prototypical heterodimeric fashion with Retinoid X Receptor (hereinafter RXR, Forman et al., Cell, 81,687-93,1995). The relevant physiological ligands of LXR seem to be oxidized derivatives of cholesterol, including 22-hydroxycholesterol and 24,25(S)-epoxycholesterol (Lehmann, et al., Biol. Chem. 272 (6), 3137-40,1997). The oxysterol ligands bound to LXR were found to regulate the expression of several genes that participate in cholesterol metabolism (Janowski, et al., Nature, 383, 728-31, 1996).

LXR is proposed to be a hepatic oxysterol sensor. Upon activation (e.g. binding of oxysterols) it influences the conversion of dietary cholesterol into bile acids by upregulating the transcription of key genes which are involved in bile acid synthesis such as CYP7A1. Hence, activation of LXR in the liver could result in an increased synthesis of bile acids from cholesterol which could lead to decreased levels of hepatic cholesterol. This proposed LXR function in hepatic cholesterol metabolism was experimentally confirmed using knockout mice. Mice lacking the receptor LXRa lost their ability to respond normally to an increase in dietary cholesterol and did not induce transcription of the gene encoding CYP7A1. This resulted in accumulation of large quantities of cholesterol in the livers and impaired hepatic function (Peet, et al., Cell, 93, 693-704, 1998).

Besides its important function in liver, LXR plays an important role in the regulation of cholesterol homeostasis in macrophages and intestinal mucosa cells where it upregulates cholesterol transporters from the ABC (=ATP binding cassette) family of membrane proteins (Repa, et al., J Biol Chem. 2002 May 24; 277 (21): 18793-800).

These transporters are believed to be crucially involved in the uptake of cholesterol from the diet since mutations in their genes leads to diseases such as sitosterolemia (Berge, et al., Science (2000); 290 (5497): 1771-5.).

Other members of the ABC transporter family seem to be responsible for the efflux of cholesterol from loaded macrophages, a process which is thought to prevent the generation of atherosclerotic lesions. Stimulation of LXR by synthetic ligands might result in an increased cholesterol efflux from macrophages and a decreased building up of cholesterol loaded atherosclerotic plaques (Venkateswaran, et al., PNAS (2000) 24; 97 (22): 12097-102; Sparrow, et al., J Biol Chem (2002) 277 (12): 10021-7; Joseph, et al., PNAS (2002); 99 (11): 7604-9). Direct evidence that synthetic LXR ligands inhibit the development of atherosclerosis has been provided in two animal models of atherosclerosis : A significant reduction in the formation of atherosclerotic plaques were shown in two studies in animal models using full LXR agonists Joseph et al. PNAS (2002) 99: 7604-9 and Terasaka et al. (2003) Terasaka et al. FEBS Lett. (2003) 536: 6-11. In addition, two recent reports have highlighted the potential use of LXR agonists in diabetes (Cao et al., (2003) J Biol Chem. 278: 1131-6 and inflammatory disorders (Joseph et al., (2003) Nat Med. 9: 213-9.

However, in animal studies it was observed that activation of LXR in the liver by full agonists like T0901317 does not only increase bile acid synthesis but also stimulates the de novo synthesis of fatty acids and triglycerids through the upregulation of key enzymes such as Fatty Acid Synthase (FAS) or Stearyl-CoA Desaturase (SCD-1) (Schultz, et al., Genes Dev (2000) 14 (22): 2831-8). Elevation of serum triglyceride levels is an independent risk factor for atherosclerosis (for review see Miller (1999) Hosp Pract (Off Ed) 34: 67-73.).

Thus, LXR activity needs to be selectively modulated for therapeutic benefit. In particular, compounds need to be found that stimulate reverse cholesterol transport, but do not significantly increase trigclyceride levels. This might be particular relevant for the usage of such compounds in diabetic patients since a even more severe lopogenic effect was reported for the full agonist T0901317 in db/db mice which serve as an animal model for diabetes (Chisholm et al. (2003) J. Lipid Res (epub August 16)).

Therefore, an ideal synthetic LXR binding compound should have properties that retain the agonistic activity on hepatic bile acid formation and ABC-transporter mediated decrease in cholesterol uptake from the diet and increased cholesterol efflux from macrophages. In parallel such a compound should lack the hyperlipidemic potential which is exerted through increased fatty acid and triclyceride synthesis.

To date only few compounds have been described which bind the LXR receptor and thus show utility for treating diseases or conditions which are due to or influenced by said nuclear receptor (Collins, et al., J Med Chem. (2002) 45 (10): 1963-6; Schutz, et al., Genes Dev (2000) 14 (22): 2831-8; Sparrow, et al., J Biol Chem (2002) 277 (12): 10021-7). No non-steroidal compounds have so far been described which show selectivity regarding the induction of ABC transporter genes without simultaneous induction of lipogenic genes like FAS and SREBP-1c (Kaneko et al. (2003) J Biol Chem (epub July 7).

It is thus an object of the invention to provide for compounds which by means of binding the LXR receptor act as partial agonists of said receptor with a selective property regarding the upregulation of genes like the ABC transporters in macrophages and/or other cell types and astronlgy reduced liability to increase the expression of genes involved in triglyceride synthetic pathways (like FAS and SREBP-1 c). These compounds should show utility for treating diseases or conditions which are due to or influenced by said nuclear receptor.

It is further an object of the invention to provide for compounds that may be used for the manufacture of a medicament for the treatment of cholesterol associated conditions or diseases. It is still a further object of the invention to provide for compounds that lower serum cholesterol and/or increase High Density Lipoproteins (HDL) and/or decrease Low Density Lipoproteins (LDL). It is also an object of the invention to provide for compounds that may be used for the treatment of lipid disorders including hypercholesterolemia, atherosclerosis, Alzheimer's disease, skin disorders, inflammation, obesity and diabetes.

### SUMMARY OF THE INVENTION

The present invention provides, inter alia, novel LXR nuclear receptor protein binding compounds according to the general formula (I) shown below. Said compounds are also binders of mammalian homologues of said receptor. Further the object of the invention was solved by providing for amongst the LXR nuclear receptor protein binding compounds according to the general formula (I) such compounds which act as partial agonists or mixed agonists/antagonists of the human LXR receptor or a mammalian homologue thereof. Further the object of the invention was solved by providing for amongst the LXR receptor protein binding compounds according to the general formula (I) such compounds which act as partial agonists of the human LXR receptor resulting therefore in the induction of ABC transporter proteins such as ABCA1 or ABCG1 in cell types such as macrophages but lacking a strong potential to induce genes involved in triglyceride synthetic pathways such as fatty acid synthase (FAS) or SREBP1c.

The invention provides for LXR agonists that may be used for the manufacture of a medicament for the treatment of cholesterol associated conditions or diseases. In a preferred embodiment compounds are provided that lower serum cholesterol and/or increase High Density lipoproteins (HDL) and/or decrease Low Density Lipoproteins (LDL). Also compounds are provided that may be used for the treatment of lipid disorders including hypercholesterolemia, atherosclerosis, Alzheimer's disease, skin disorders, inflammation, obesity and diabetes.

The foregoing merely summarizes certain aspects of the present invention and is not intended, nor should it be construed, to limit the invention in any manner. All patents and other publications recited herein are hereby incorporated by reference in their entirety.

### DETAILED DESCRIPTION OF THE PREFERRED EMBODIMENTS

The invention provides for a compound of the formula (I), or pharmaceutical acceptable salts or solvates thereof, hereinafter also referred to as the “compounds according to the invention” including particular and preferred embodiments thereof.

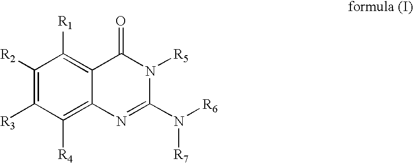

In one embodiment of the invention in formula (I) above R₁, R₂, R₃ and/or R₄, is independently from each other selected from H, halogen, hydroxy, protected hydroxy, cyano, nitro, C₁ to C₆ alkyl, C₁ to C₆ substituted alkyl, C₁ to C₇ alkoxy, C₁ to C₇ substituted alkoxy, C₁ to C₇ acyl, C₁ to C₇ substituted acyl, C₁ to C₇ acyloxy, carboxy, protected carboxy, carboxymethyl, protected carboxymethyl, hydroxymethyl, protected hydroxymethyl, amino, protected amino, (monosubstituted) amino, protected (monosubstituted) amino, (disubstituted) amino, carboxamide, protected carboxamide, N—(C₁ to C₆ alkyl)carboxamide, protected N—(C₁ to C₆ alkyl)carboxamide, N,N-di(C₁ to C₆ alkyl)carboxamide, trifluoromethyl, N—((C₁ to C₆ alkyl)sulfonyl)amino, N-(phenylsulfonyl)amino or phenyl, wherein the phenyl is substituted or unsubstituted, such that, for example, a biphenyl results. R5 is H, C₁ to C₈ alkyl, C₁ to C₈ substituted alkyl, C₇ to C₁₂ alkylphenyl or C₇ to C₁₂ substituted phenylalkyl, R6 is H, C₁ to C₈ alkyl, C₁ to C₈ substituted alkyl, C₇ to C₁2 alkylphenyl or C₇ to C₁2 substituted phenylalkyl, R7 is H, C₁ to C₈ alkyl, C₁ to C₈ substituted alkyl, C₇ to C₁₂ alkylphenyl or C₇ to C₁₂ substituted phenylalkyl, and R₆ and R₇ may be taken together with nitrogen to form a heterocycle or substituted heterocycle or a heteroaryl or substituted heteroaryl ring.

The compounds of the invention can also exist as solvates and hydrates. Thus, these compounds may crystallize with, for example, waters of hydration, or one, a number of, or any fraction thereof of molecules of the mother liquor solvent. The solvates and hydrates of such compounds are included within the scope of this invention.

In one particular embodiment of the invention in formula (I) above, namely formula (Ia)

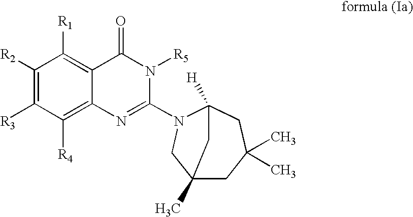

R₁, R₂, R₃, R₄ are independently from each other selected from H, halogen, hydroxy, protected hydroxy, cyano, nitro, C₁ to C₆ alkyl, C₁ to C₆ substituted alkyl, C₁ to C₇ alkoxy, C₁ to C₇ substituted alkoxy, C₁ to C₇ acyl, C₁ to C₇ substituted acyl, C₁ to C₇ acyloxy, carboxy, protected carboxy, carboxymethyl, protected carboxymethyl, hydroxymethyl, protected hydroxymethyl, amino, protected amino, (monosubstituted) amino, protected (monosubstituted) amino, (disubstituted) amino, carboxamide, protected carboxamide, N—(C₁ to C₆ alkyl)carboxamide, protected N—(C₁ to C₆ alkyl) carboxamide, N,N-di(C₁ to C₆ alkyl)carboxamide, trifluoromethyl, N—((C₁ to C₆ alkyl)sulfonyl)amino, N-(phenylsulfonyl)amino or phenyl, and R₅ is H, C₁ to C₈ alkyl, C₁ to C₈ substituted alkyl, C₇ to C₁₂ alkylphenyl or C₇ to C₁₂ substituted phenylalkyl.

The symbol “H” denotes a hydrogen atom.

The term “C₁ to C₇ acyl” encompasses groups such as formyl, acetyl, propionyl, butyryl, pentanoyl, pivaloyl, hexanoyl, heptanoyl, benzoyl and the like. Preferred acyl groups are acetyl and benzoyl.

The term “C₁ to C₇ substituted acyl” denotes the acyl group substituted by one or more, and preferably one or two, halogen, hydroxy, protected hydroxy, oxo, protected oxo, cyclohexyl, naphthyl, amino, protected amino, (monosubstituted) amino, protected (monosubsti-tuted) amino, (disubstituted) amino, guanidino, heterocyclic ring, substituted heterocyclic ring, imidazolyl, indolyl, pyrrolidinyl, C₁ to C₇ alkoxy, C₁ to C₇ acyl, C₁ to C₇ acyloxy, nitro, C₁ to C₆ alkyl ester, carboxy, protected carboxy, carbamoyl, carboxamide, protected carboxamide, N—(C₁ to C₆ alkyl)carboxamide, protected N—(C₁ to C₆ alkyl)carboxamide, N,N-di(C₁ to C₆ alkyl)carboxamide, cyano, methylsulfonylamino, thiol, C₁ to C₄ alkylthio or C₁ to C₄ alkylsulfonyl groups or 2,3 or 4-fluorophenyl and the like; a mono or di(hydroxy)phenyl group such as 2,3 or 4-hydroxyphenyl, 2,4-dihydroxyphenyl, the protected-hydroxy derivatives thereof and the like; a nitrophenyl group such as 2,3 or 4-nitrophenyl; a cyanophenyl group, for example, 2,3 or 4-cyanophenyl; a mono- or di(alkyl)phenyl group such as 2,3 or 4methylphenyl, 2,4-dimethylphenyl, 2,3 or 4-(iso-propyl)phenyl, 2,3 or 4-ethylphenyl, 2,3 or 4-(n-propyl)phenyl and the like ; a mono or di(alkoxyl)phenyl group, for example, 2,6-dimethoxyphenyl, 2,3 or 4-methoxyphenyl, 2,3 or 4-ethoxyphenyl, 2,3 or 4-(isopropoxy)phenyl, 2,3 or 4-(t-butoxy)phenyl, 3-ethoxy-4-methoxyphenyl and the like; 2,3 or 4-trifluoromethylphenyl; a mono- or dicarboxyphenyl or (protected carboxy)phenyl group such as 2,3 or 4-carboxyphenyl or 2,4-di(protected carboxy)phenyl; a mono-or di(hydroxymethyl)phenyl or (protected hydroxymethyl)phenyl such as 2,3 or 4-(protected hydroxymethyl)phenyl or 3,4-di(hydroxymethyl)phenyl; a mono-or di(aminomethyl)phenyl or (protected aminomethyl)phenyl such as 2,3 or 4-(aminomethyl)phenyl or 2,4-(protected aminomethyl)phenyl; or a mono-or di(N-(methylsulfonylamino))phenyl such as 2,3 or 4-(N-(methylsulfonylamino))phenyl. Also, the term “substituted phenyl” represents disubstituted phenyl groups wherein the substituents are different, for example, 3-methyl-4-hydroxyphenyl, 3-chloro-4-hydroxyphenyl, 2-methoxy-4-bromophenyl, 4-ethyl-2-hydroxyphenyl, 3-hydroxy-4-nitrophenyl, 2-hydroxy 4-chlorophenyl and the like. The substituted acyl groups may be substituted once or more, and preferably once or twice, with the same or with different substituents.

The term “substituted phenyl” specifies a phenyl group substituted with one or more, and preferably one or two, moieties chosen from the groups consisting of halogen, hydroxy, protected hydroxy, cyano, nitro, C₁ to C₆ alkyl, C₁ to C₆ substituted alkyl, C₁ to C₇ alkoxy, C₁ to C₇ substituted alkoxy, C₁ to C₇ acyl, C₁ to C₇ substituted acyl, C₁ to C₇ acyloxy, carboxy, protected carboxy, carboxymethyl, protected carboxymethyl, hydroxymethyl, protected hydroxymethyl, amino, protected amino, (monosubstituted) amino, protected (monosubstituted) amino, (disubstituted) amino, carboxamide, protected carboxamide, N—(C₁ to C₆ alkyl)carboxamide, protected N—(C₁ to C₆ alkyl)carboxamide, N,N-di(C₁ to C₆ alkyl)carboxamide, trifluoromethyl, N—((C₁ to C₆ alkyl)sulfonyl)amino, N-(phenylsulfonyl)amino or phenyl, wherein the phenyl is substituted or unsubstituted, such that, for example, a biphenyl results.

Examples of the term “substituted phenyl” include a mono-or di(halo)phenyl group such as 2,3 or 4-chlorophenyl, 2,6-dichlorophenyl, 2,5-dichlorophenyl, 3,4-dichlorophenyl, 2,3 or 4-bromophenyl, 3,4-dibromophenyl, 3-chloro-4-fluorophenyl, 2,3 or 4-fluorophenyl and the like; a mono or di(hydroxy)phenyl group such as 2,3 or 4-hydroxyphenyl, 2,4-dihydroxyphenyl, the protected-hydroxy derivatives thereof and the like; a nitrophenyl group such as 2,3 or 4-nitrophenyl; a cyanophenyl group, for example, 2,3 or 4-cyanophenyl; a mono- or di(alkyl)phenyl group such as 2,3 or 4-methylphenyl, 2,4-dimethylphenyl, 2,3 or 4-(iso-propyl)phenyl, 2,3 or 4-ethylphenyl, 2,3 or 4-(n-propyl)phenyl and the like; a mono or d (alkoxyl phenyl group, for example, 2,6-dimethoxyphenyl, 2,3 or 4-methoxyphenyl, 2,3 or 4-ethoxyphenyl, 2,3 or 4-(isopropoxy)phenyl, 2,3 or 4-(t-butoxy)phenyl, 3-ethoxy-4-methoxyphenyl and the like; 2,3 or4-trifluoromethylphenyl; a mono- or dicarboxyphenyl or (protected carboxy)phenyl group such as 2,3 or 4-carboxyphenyl or 2,4-di(protected carboxy)phenyl; a mono- or di(hydroxymethyl)phenyl or (protected hydroxymethyl)phenyl such as 2,3 or 4-(protected hydroxymethyl)phenyl or 3,4-di(hydroxymethyl)phenyl; a mono- or di(aminomethyl)phenyl or (protected aminomethyl)phenyl such as 2,3 or 4-(aminomethyl)phenyl or 2,4-(protected aminomethyl)phenyl; or a mono- or di(N-(methylsulfonylamino))phenyl such as 2,3 or 4-(N-(methylsulfonylamino))phenyl. Also, the term “substituted phenyl” represents disubstituted phenyl groups wherein the substituents are different, for example, 3-methyl-4-hydroxyphenyl, 3-chloro-4-hydroxyphenyl, 2-methoxy-4-bromophenyl, 4-ethyl-2-hydroxyphenyl, 3-hydroxy-4-nitrophenyl, 2-hydroxy-4-chlorophenyl and the like.

The term “heteroaryl” means a heterocyclic aromatic derivative which is a five-membered or six-membered ring system having from 1 to 4 heteroatoms, such as oxygen, sulfur and/or nitrogen, in particular nitrogen, either alone or in conjunction with sulfur or oxygen ring atoms.

Examples of heteroaryls include pyridinyl, pyrimidinyl, and pyrazinyl, pyridazinyl, pyrrolo, furano, thiopheno, oxazolo, isoxazolo, phthalimido, thiazol and the like.

The term “substituted heteroaryl” means the above-described heteroaryl is substituted with, for example, one or more, and preferably one or two, substituents which are the same or different which substituents can be halogen, hydroxy, protected hydroxy, cyano, nitro, C₁ to C₆ alkyl, C₁ to C₇ alkoxy, C₁ to C₇ substituted alkoxy, C₁ to C₇ acyl, C₁ to C₇ substituted acyl, C₁ to C₇ acyloxy, carboxy, protected carboxy, carboxymethyl, protected carboxymethyl, hydroxymethyl, protected hydroxymethyl, amino, protected amino, (monosubstituted)amino, protected (monosubstituted)amino, (disubstituted)amino, carboxamide, protected carboxamide, N—(C₁ to C₆ alkyl)carboxamide, protected N—(C₁ to C₆ alkyl)carboxamide, N,N-di(C₁ to C₆ alkyl)carboxamide, trifluoromethyl, N—((C₁ to C₆ alkyl)sulfonyl)amino or N-(phenylsulfonyl)amino groups.

The term “substituted naphthyl” specifies a naphthyl group substituted with one or more, and preferably one or two, moieties either on the same ring or on different rings chosen from the groups consisting of halogen, hydroxy, protected hydroxy, cyano, nitro, C₁ to C₆ alkyl, C₁ to C₇ alkoxy, C₁ to C₇ acyl, C₁ to C₇ acyloxy, carboxy, protected carboxy, carboxymethyl, protected carboxymethyl, hydroxymethyl, protected hydroxymethyl, amino, protected amino, (monosubstituted) amino, protected (monosubstituted) amino, (disubstituted) amino, carboxamide, protected carboxamide, N—(C₁ to C₆ alkyl)carboxamide, protected N—(C₁ to C₆ alkyl)carboxamide, N,N-di(C₁ to C₆ alkyl)carboxamide, trifluoromethyl, N—((C₁ to C₆ alkyl)sulfonyl)amino or N-(phenylsulfonyl)amino.

Examples of the term “substituted naphthyl” includes a mono or di(halo)naphthyl group such as 1,2,3,4,5,6,7 or 8-chloronaphthyl, 2,6-dichloronaphthyl, 2,5-dichloronaphthyl, 3,4-dichloronaphthyl, 1,2,3,4,5,6,7 or 8-bromonaphthyl, 3,4-dibromonaphthyl, 3-chloro-4-fluoronaphthyl, 1,2,3,4,5,6,7 or 8-fluoronaphthyl and the like; a mono or di(hydroxy)naphthyl group such as 1,2,3,4,5,6,7 or 8-hydroxynaphthyl, 2,4-dihydroxynaphthyl, the protected-hydroxy derivatives thereof and the like; a nitronaphthyl group such as 3- or 4-nitronaphthyl; a cyanonaphthyl group, for example, 1,2,3,4,5,6,7 or 8-cyanonaphthyl; a mono- or di(alkyl)naphthyl group such as 2,3,4,5,6,7 or 8-methylnaphthyl,1, 2,4-dimethylnaphthyl, 1,2,3,4,5,6,7 or 8-(isopropyl)naphthyl, 1,2,3,4,5,6,7 or 8-ethylnaphthyl, 1,2,3,4,5,6,7 or 8-(n-propyl)naphthyl and the like; a mono or di(alkoxy)naphthyl group, for example, 2,6-dimethoxynaphthyl, 1,2,3,4,5,6,7 or 8-methoxynaphthyl, 1,2,3,4,5,6,7 or 8-ethoxynaphthyl, 1,2,3,4,5,6,7 or 8-(isopropoxy)naphthyl, 1,2,3,4,5,6,7 or 8-(t-butoxy)naphthyl, 3-ethoxy-4-methoxynaphthyl and the like; 1,2,3,4, 5,6, 7 or 8-trifluoromethylnaphthyl; a mono- or dicarboxynaphthyl or (protected carboxy)naphthyl group such as 1,2,3,4,5,6,7 or 8-carboxynaphthyl or 2,4-di(-protected carboxy)naphthyl; a mono- or di(hydroxymethyl)naphthyl or (protected hydroxymethyl)naphthyl such as 1,2,3,4,5,6,7 or 8-(protected hydroxymethyl)naphthyl or 3,4-di(hydroxymethyl)naphthyl; a mono- or di(amino)naphthyl or (protected amino)naphthyl such as 1,2,3,4,5,6,7 or 8-(amino)naphthyl or 2,4-(protected amino)-naphthyl, a mono- or di(aminomethyl)naphthyl or (protected aminomethyl)naphthyl such as 2,3 or 4-(aminomethyl)naphthyl or 2,4-(protected aminomethyl)naphthyl; or a mono- or di-(N-methylsulfonylamino)naphthyl such as 1,2,3,4,5,6,7 or 8-(N-methylsulfonylamino)naphthyl. Also, the term “substituted naphthyl” represents disubstituted naphthyl groups wherein the substituents are different, for example, 3-methyl-4-hydroxynaphth-1-yl, 3-chloro-4-hydroxynaphth-2-yl, 2-methoxy-4-bromonaphth-1-yl, 4-ethyl-2-hydroxynaphth-1-yl, 3-hydroxy-4-nitronaphth-2-yl, 2-hydroxy-4-chloronaphth-1-yl, 2-methoxy-7-bromonaphth-1-yl, 4-ethyl-5-hydroxynaphth-2-yl, 3-hydroxy-8-nitronaphth-2-yl, 2-hydroxy-5-chloronaphth-1-yl, and the like.

The term “C₁ to C₈ alkyl” denotes such radicals as methyl, ethyl, n-propyl, isopropyl, n-butyl, iso-butyl, sec-butyl, tert-butyl, amyl, tert-amyl, hexyl, n-heptyl, 2-heptyl, 3-heptyl, 4-heptyl, 2-methyl-1-hexyl, 2-methyl-2-hexyl, 2-methyl-3-hexyl, n-octyl, and the like.

The term “C₂ to C₆ alkenyl” denotes such radicals as propenyl or butenyl.

Examples of the above substituted alkyl groups include the 2-oxo-prop-1-yl,3-oxo-but-1-yl, cyanomethyl, nitromethyl, chloromethyl, hydroxymethyl, tetrahydropyranyloxymethyl, trityloxymethyl, propionyloxymethyl, amino, methylamino, aminomethyl, dimethylamino, carboxymethyl, allyloxycarbonylmethyl, allyloxycarbonylaminomethyl, methoxymethyl, ethoxymethyl, t-butoxymethyl, acetoxymethyl, chloromethyl, bromomethyl, iodomethyl, trifluoromethyl, 6-hydroxyhexyl, 2,4-dichloro(n-butyl), 2-aminopropyl, 1-chloroethyl, 2-chloroethyl, 1-bromoethyl, 2-chloroethyl, 1-fluoroethyl, 2-fluoroethyl, 1-iodoethyl, 2-iodoethyl, 1-chloropropyl, 2-chloropropyl, 3-chloropropyl, 1-bromopropyl, 2-bromopropyl, 3-bromopropyl, 1-fluoropropyl, 2-fluoropropyl, 3-fluoropropyl, 1-iodopropyl, 2-iodopropyl, 3-iodopropyl, 2-aminoethyl, 1-aminoethyl, N-benzoyl-2-aminoethyl, N-acetyl-2-aminoethyl, N-benzoyl-1-aminoethyl, N-acetyl-1-aminoethyl, and the like.

The term “C₁ to C₈ substituted alkyl” denotes that the above C₁ to C₈ alkyl groups are substituted by one or more, and preferably one or two, halogen, hydroxy, protected hydroxy, oxo, protected oxo, C₃ to C₇ cycloalkyl, naphthyl, amino, protected amino, (monosubstituted) amino, protected (monosubstituted) amino, (disubstituted) amino, guanidino, protected guanidino, heterocyclic ring, substituted heterocyclic ring, imidazolyl, indolyl, pyrrolidinyl, C₁ to C₇ alkoxy, C₁ to C₇ acyl, C₁ to C₇ acyloxy, nitro, carboxy, protected carboxy, carbamoyl, carboxamide, protected carboxamide, N—(C₁ to C₆ alkyl)carboxamide, protected N—(C₁ to C₆ alkyl)carboxamide, N,N-di(C₁ to C₆ alkyl)carboxamide, cyano, methylsulfonylamino, thiol, C₁ to C₄ alkylthio or C₁ to C₄ alkylsulfonyl groups. The substituted alkyl groups may be substituted once or more, and preferably once or twice, with the same or with different substituents.

The term “C₇ to C₁₂ phenylalkyl” denotes a C₁ to C₆ alkyl group substituted at any position by a phenyl, substituted phenyl, heteroaryl or substituted heteroaryl. Examples of such a group include benzyl, 2-phenylethyl, 3-phenyl(n-propyl), 4-phenylhexyl, 3-phenyl(n-amyl), 3-phenyl(sec-butyl) and the like. Preferred C₇ to C₁2 phenylalkyl groups are the benzyl and the phenylethyl groups.

The term “C₇ to C₁₂ substituted phenylalkyl” denotes a C₇ to C₁₂ phenylalkyl group substituted on the C₁ to C₆ alkyl portion with one or more, and preferably one or two, groups chosen from halogen, hydroxy, protected hydroxy, oxo, protected oxo, amino, protected amino, (monosubstituted) amino, protected (monosubstituted) amino, (disubstituted) amino, guanidino, protected guanidino, heterocyclic ring, substituted heterocyclic ring, C₁ to C₆ alkyl, C₁ to C₆ substituted alkyl, C₁ to C₇ alkoxy, C₁ to C₇ substituted alkoxy, C₁ to C₇ acyl, C₁ to C₇ substituted acyl, C₁ to C₇ acyloxy, nitro, carboxy, protected carboxy, carbamoyl, carboxamide, protected carboxamide, N—(C₁ to C₆ alkyl)carboxamide, protected N—(C₁ to C₆ alkyl)carboxamide, N,N—(C₁ to C₆ dialkyl)carboxamide, cyano, N—(C₁ to C₆ alkylsulfonyl)amino, thiol, C₁ to C₄ alkylthio, C₁ to C₄ alkylsulfonyl groups; and/or the phenyl group may be substituted with one or more, and preferably one or two, substituents chosen from halogen, hydroxy, protected hydroxy, cyano, nitro, C₁ to C₆ alkyl, C₁ to C₆ substituted alkyl, C₁ to C₇ alkoxy, C₁ to C₇ substituted alkoxy, C₁ to C₇ acyl, C₁ to C₇ substituted acyl, C₁ to C₇ acyloxy, carboxy, protected carboxy, carboxymethyl, protected carboxymethyl, hydroxymethyl, protected hydroxymethyl, amino, protected amino, (monosubstituted) amino, protected (monosubstituted) amino, (disubstituted) amino, carboxamide, protected carboxamide, N—(C₁ to C₆ alkyl)carboxamide, protected N—(C₁ to C₆ alkyl)carboxamide, N,N-di(C₁ to C₆ alkyl)carboxamide, trifluoromethyl, N—((C₁ to C₆ alkyl)sulfonyl)amino, N-(phenylsulfonyl)amino, cyclic C₂ to C₇ alkylene or a phenyl group, substituted or unsubstituted, for a resulting biphenyl group. The substituted alkyl or phenyl groups may be substituted with one or more, and preferably one or two, substituents which can be the same or different.

Examples of the term “C₇ to C₁₂ substituted phenylalkyl” include groups such as 2-phenyl-1-chloroethyl, 2-(4-methoxyphenyl)ethyl, 4-(2,6-dihydroxyphenyl)n-hexyl, 2-(5-cyano-3-methoxyphenyl)n-pentyl, 3-(2,6-dimethylphenyl)n-propyl, 4-chloro-3-aminobenzyl, 6-(4-methoxyphenyl)-3-carboxy(n-hexyl), 5-(4-aminomethylphenyl)-3-(aminomethyl)n-pentyl, 5-phenyl-3-oxo-n-pent-1-yl, and the like.

As outlined above, R₆ and R₇ may be taken together with nitrogen to form a heterocycle or substituted heterocycle of the following kind aziridine, azetidine, pyrrolidine, 3-methylpyrrolidine, 3-aminopyrrolidine, 3-hydroxypyrrolidine, pyrazolidine, imidazolidine, piperidine, 2-methylpiperidine, piperazine, morpholine, azepine, or tetrahydroisoquinoline.

The term “heterocycle” or “heterocyclic ring” denotes optionally substituted five-membered to eight-membered rings that have 1 to 4 heteroatoms, such as oxygen, sulfur and/or nitrogen, in particular nitrogen, either alone or in conjunction with sulfur or oxygen ring atoms. These five-membered to eight-membered rings may be saturated, fully unsaturated or partially unsaturated, with fully saturated rings being preferred. Preferred heterocyclic rings include morpholino, piperidinyl, piperazinyl, 2-amino-imidazoyl, tetrahydrofurano, pyrrolo, tetrahydrothiophen-yl, hexylmethyleneimino and heptylmethyleneimino.

The term “substituted heterocycle” or “substituted heterocyclic ring” means the above-described heterocyclic ring is substituted with, for example, one or more, and preferably one or two, substituents which are the same or different which substituents can be halogen, hydroxy, protected hydroxy, cyano, nitro, C₁ to C₁₂ alkyl, C₁ to C₁₂ alkoxy, C₁ to C₁₂ substituted alkoxy, C₁ to C₁₂ acyl, C₁ to C₁₂ acyloxy, carboxy, protected carboxy, carboxymethyl, protected carboxymethyl, hydroxymethyl, protected hydroxymethyl, amino, protected amino, (monosubstituted) amino, protected (monosubstituted) amino, (disubstituted) amino carboxamide, protected carboxamide, N—(C₁ to C₁₂ alkyl) carboxamide, protected N—(C₁ to C₁₂ alkyl)carboxamide, N,N-di(C₁ to C₁₂ alkyl)carboxamide, trifluoromethyl, N—((C₁ to C₁₂ alkyl)sulfonyl)amino, N-(phenylsulfonyl)amino, heterocycle or substituted heterocycle groups.

The term “C₁ to C₈ alkoxy” as used herein denotes groups such as methoxy, ethoxy, n-propoxy, isopropoxy, n-butoxy, t-butoxy and like groups. A preferred alkoxy is methoxy. The term “C₁ to C₈ substituted alkoxy” means the alkyl portion of the alkoxy can be substituted in the same manner as in relation to C₁ to C₈ substituted alkyl.

The term “C₁ to C₈ aminoacyl” encompasses groups such as formyl, acetyl, propionyl, butyryl, pentanoyl, pivaloyl, hexanoyl, heptanoyl, octanoyl, benzoyl and the like.

The term “C₁ to C₈ substituted aminoacyl” denotes the acyl group substituted by one or more, and preferably one or two, halogen, hydroxy, protected hydroxy, oxo, protected oxo, cyclohexyl, naphthyl, amino, protected amino, (monosubstituted) amino, protected (monosubstituted) amino, (disubstituted) amino, guanidino, heterocyclic ring, substituted heterocyclic ring, imidazolyl, indolyl, pyrrolidinyl, C₁ to C₁₂ alkoxy, C₁ to C₁₂ acyl, C₁ to C₁₂ acyloxy, nitro, C₁ to C₁₂ alkyl ester, carboxy, protected carboxy, carbamoyl, carboxamide, protected carboxamide, N—(C₁ to C₁₂ alkyl)carboxamide, protected N—(C₁ to C₁₂ alkyl)carboxamide, N,N-di(C₁ to C₁₂ alkyl)carboxamide, cyano, methylsulfonylamino, thiol, C₁ to C₁₀ alkylthio or C₁ to C₁₀ alkylsulfonyl groups. The substituted acyl groups may be substituted once or more, and preferably once or twice, with the same or with different substituents.

Examples of C₁ to C₈ substituted acyl groups include 4-phenylbutyroyl, 3-phenylbutyroyl, 3-phenylpropanoyl, 2-cyclohexanylacetyl, cyclohexanecarbonyl, 2-furanoyl and 3-dimethylaminobenzoyl.

This invention provides a pharmaceutical composition comprising an effective amount of a compound according to the invention. Such compounds can be administered by various routes, for example oral, subcutaneous, intramuscular, intravenous or intracerebral. The preferred route of administration would be oral at daily doses of the compound for adult human treatment of about 0.01-5000 mg, preferably 1-1500 mg per day. The appropriate dose may be administered in a single dose or as divided doses presented at appropriate intervals for example as two, three four or more subdoses per day.

For preparing pharmaceutical compositions containing compounds of the invention, inert, pharmaceutically acceptable carriers are used. The pharmaceutical carrier can be either solid or liquid. Solid form preparations include, for example, powders, tablets, dispersible granules, capsules, cachets, and suppositories.

A solid carrier can be one or more substances which can also act as diluents, flavoring agents, solubilizers, lubricants, suspending agents, binders, or tablet disintegrating agents; it can also be an encapsulating material.

In powders, the carrier is generally a finely divided solid which is in a mixture with the finely divided active component. In tablets, the active compound is mixed with the carrier having the necessary binding properties in suitable proportions and compacted in the shape and size desired.

For preparing pharmaceutical composition in the form of suppositories, a low-melting wax such as a mixture of fatty acid glycerides and cocoa butter is first melted and the active ingredient is dispersed therein by, for example, stirring. The molten homogeneous mixture is then poured into convenient-sized molds and allowed to cool and solidify.

Powders and tablets preferably contain between about 5% to about 70% by weight of the active ingredient. Suitable carriers include, for example, magnesium carbonate, magnesium stearate, talc, lactose, sugar, pectin, dextrin, starch, tragacanth, methyl cellulose, sodium carboxymethyl cellulose, a low-melting wax, cocoa butter and the like.

The pharmaceutical compositions can include the formulation of the active compound with encapsulating material as a carrier providing a capsule in which the active component (with or without other carriers) is surrounded by a carrier, which is thus in association with it. In a similar manner, cachets are also included. Tablets, powders, cachets, and capsules can be used as solid dosage forms suitable for oral administration.

Liquid pharmaceutical compositions include, for example, solutions suitable for oral or parenteral administration, or suspensions, and emulsions suitable for oral administration. Sterile water solutions of the active component or sterile solutions of the active component in solvents comprising water, ethanol, or propylene glycol are examples of liquid compositions suitable for parenteral administration. Sterile solutions can be prepared by dissolving the active component in the desired solvent system, and then passing the resulting solution through a membrane filter to sterilize it or, alternatively, by dissolving the sterile compound in a previously sterilized solvent under sterile conditions.

In one embodiment of the present invention a compound is claimed according to formula (1) above, or pharmaceutical acceptable salts or solvates thereof, wherein R₁, R₂, R₃, R₄ is H, halogen, hydroxy, protected hydroxy, cyano, nitro, C₁ to C₆ alkyl, C₁ to C₆ substituted alkyl, C₁ to C₇ alkoxy, C₁ to C₇ substituted alkoxy, C₁ to C₇ acyl, C₁ to C₇ substituted acyl, C₁ to C₇ acyloxy, carboxy, protected carboxy, carboxymethyl, protected carboxymethyl, hydroxymethyl, protected hydroxymethyl, amino, protected amino, (monosubstituted) amino, protected (monosubstituted) amino, (disubstituted) amino, carboxamide, protected carboxamide, N—(C₁ to C₆ alkyl)carboxamide, protected N—(C₁ to C₆ alkyl)carboxamide, N,N-di(C₁ to C₆ alkyl)carboxamide, trifluoromethyl, N—((C₁ to C₆ alkyl)sulfonyl)amino, N-(phenylsulfonyl)amino or phenyl, R5 is H, C₁ to C₈ alkyl, C₁ to C₈ substituted alkyl, C₇ to C₁₂ alkylphenyl or C₇ to C₁₂ substituted phenylalkyl, R₆ and R₇ may be taken together with nitrogen to form the heterocycle according to formula (2).

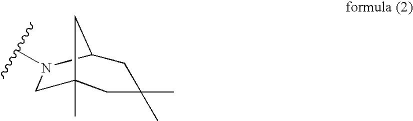

In a preferred embodiment of the invention a compound is provided, or pharmaceutical acceptable salts or solvates thereof, wherein R₁, R₂, R₃, R₄, is H, halogen, hydroxy, protected hydroxy, cyano, nitro, C₁ to C₆ alkyl, C₁ to C₆ substituted alkyl, C₁ to C₇alkoxy, C₁ to C₇ substituted alkoxy, C₁ to C₇ acyl, C₁ to C₇ substituted acyl, C₁ to C₇ acyloxy, carboxy, protected carboxy, carboxymethyl, protected carboxymethyl, hydroxymethyl, protected hydroxymethyl, amino, protected amino, (monosubstituted) amino, protected (monosubstituted) amino, (disubstituted) amino, carboxamide, protected carboxamide, N—(C₁ to C₆ alkyl)carboxamide, protected N—(C₁ to C₆ alkyl)carboxamide, N,N-di(C₁ to C₆ alkyl)carboxamide, trifluoromethyl, N—((C₁ to C₆ alkyl)sulfonyl)amino, N-(phenylsulfonyl)amino or phenyl, R₅ is H, C₁ to C₈ alkyl, C₁ to C₈ substituted alkyl, R₆ and R₇ may be taken together with nitrogen to form the heterocycle according to formula (2) shown above.

The inventors have been able to demonstrate that the compound according to formula (4) has a low effective concentration at LXR with an EC₅₀ of 0.5 uM wherein the EC₅₀ reflects the half-maximal effective concentration, and which is higher than the EC₅₀ of 0.015 uM for the published LXR agonist T0901317 (J. Schultz et al., Genes Dev. 14, 2831-2838, 2000).

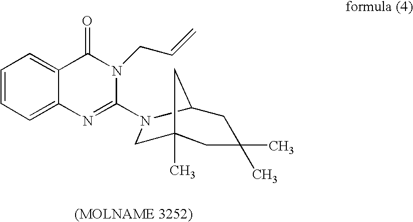

The inventors have also found the compounds according to formula (5, 6, and 7) (shown below) to be active as agonist of the LXR human nuclear receptor (see figures for details).

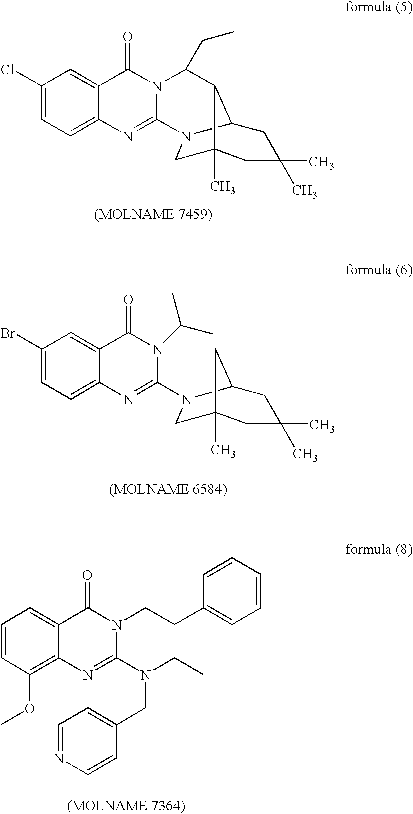

In a preferred embodiment of the invention in the compounds claimed, or the pharmaceutical acceptable salts or solvates thereof, R₁, R₃, and R₄ are H, R₂ is halogen and preferably iodine over bromine and chlorine and R₅ is H, C₁ to C₈ alkyl or C₁ to C₈ substituted alkyl.

Another preferred compound according to the invention has the following formula (8a)

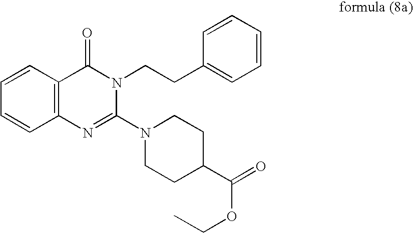

A particularly preferred compound which may act as a partial agonist of LXR is shown in formula (9) below (MOLNAME TR1040001892). It has been demonstrated that this compound has a low effective concentration at LXR with an EC₅₀ of 2 uM in a FRET assay wherein the EC₅₀ reflects the half-maximal effective concentration, and which is higher than the EC₅₀ of 0.015 uM for the published LXR agonist T0901317 (J. Schultz et al., Genes Dev. 14, 2831-2838, 2000). The compound according to formula (9) does show selective upregulation of ABCA1 and ABCG1 in THP-1 macrophages but does not significantly upregulate FAS and much reduced SREBP-1c in HepG2 cells (see EXAMPLE 5 further down).

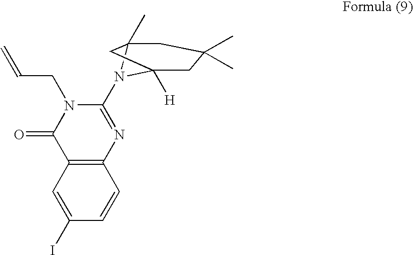

It has also been found that the compound according to formula (10) (Molname TR1040011382) to be active as partial agonist of the LXR human nuclear receptors with a selective upregulation of the target genes ABCA1 and ABCG1 in THP-1 cells compared to FAS and SREBP-1 c in HepG2 cells.

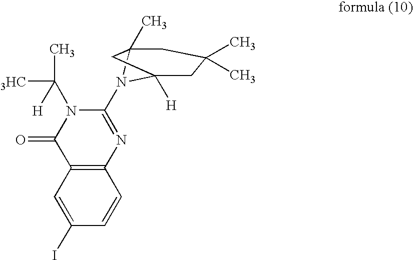

It has also been found that the compounds according to formulas (11) (Molname TR104000221 1) and (12) (Molname TR1040002212) to be active as partial agonist of the LXR human nuclear receptors however with a reduced selectivity regarding the upregulation of the target genes ABCA1 and ABCG1 in THP-1 cells versus FAS and SREBP-1c in HepG2 cells compared to compounds of formula (9) and (10) (see EXAMPLE 5 further down)

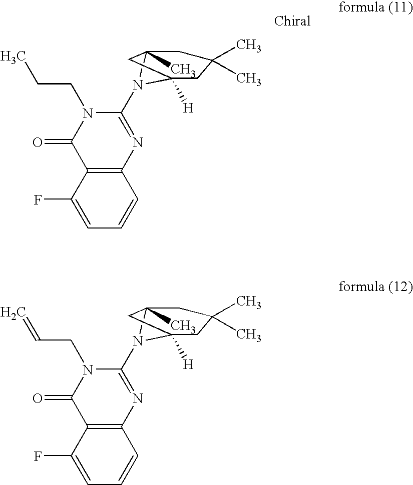

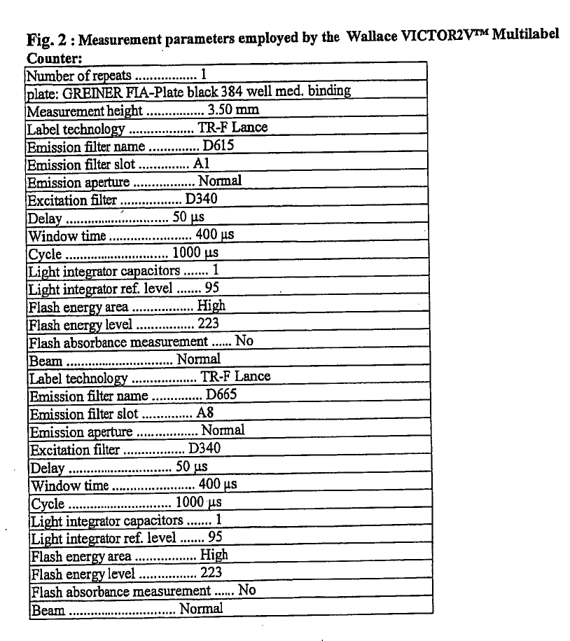

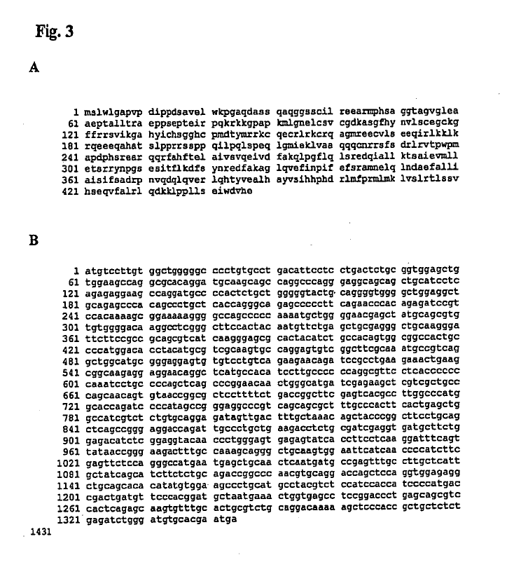

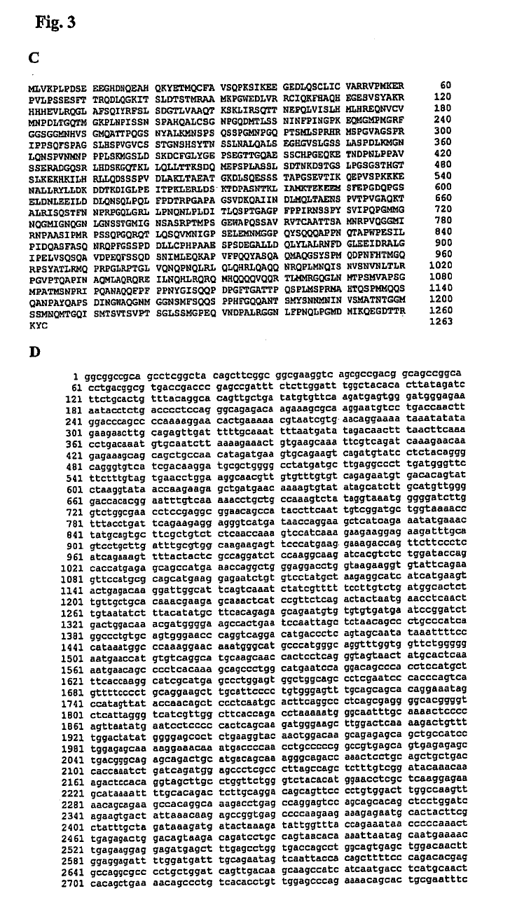

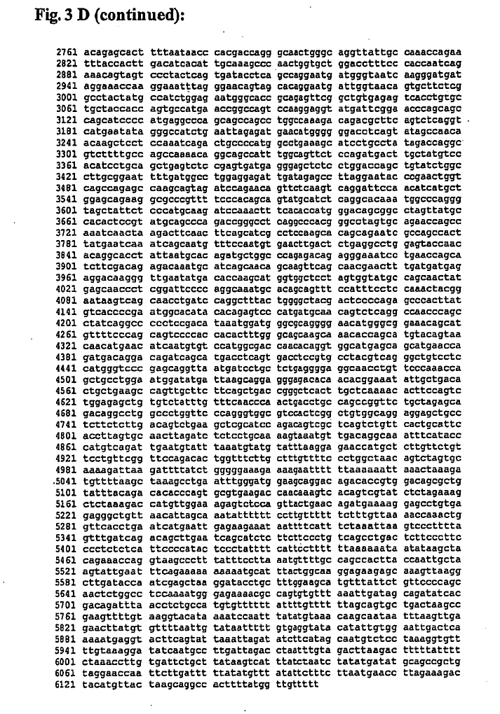

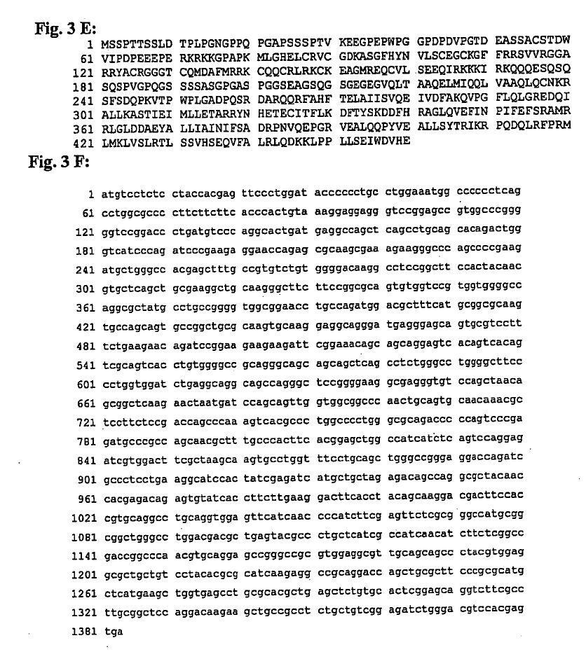

In particular the invention relates to a compound as described above wherein said compounds is capable of binding the LXR receptor protein or a portion thereof encoded by a nucleic acid according to SEQ ID NO:1 or NO: 2 (FIG. 3) or a mammalian homologue thereof. This compound can bind to the LXR receptor protein or a portion thereof in a mixture comprising 10-200 ng of LXR receptor protein, a fusion protein containing LXR or a portion thereof, preferably the ligand binding domain, fused to a Tag, 5-100 mM Tris/HCl at pH 6.8-8.3; 60-1000 mM KCl; 0-20 mM MgCl₂; 100-1000 ng/ul BSA in a total volume of preferably about 25 ul (see also EXAMPLE 1 and FIG. 2).

A mammalian receptor protein homologue of the protein encoded by a nucleic acid according to SEQ ID NO: 1 or SEQ ID NO: 2, as used herein is a protein that performs substantially the same task as LXR does in humans and shares at least 40% sequence identity at the amino acid level, preferably over 50% sequence identity at the amino acid level more preferably over 65% sequence identity at the amino acid level, even more preferably over 75% sequence identity at the amino acid level and most preferably over 85% sequence identity at the amino acid level.

The invention in particular concerns a method for prevention or treatment of a LXR receptor protein or LXR receptor protein homologue mediated disease or condition in a mammal comprising administration of a therapeutically effective amount of a compound according to the invention wherein the prevention or treatment is directly or indirectly accomplished through the binding of a compound according to the invention to the LXR receptor protein or to the LXR receptor protein homologue.

The term “mediated” herein means that the physiological pathway in which the LXR receptor protein acts is either directly or indirectly involved in the disease or condition to be treated or prevented. In the case where it is indirectly involved it could be that, e. g. modulating the activity of LXR by a compound according to the invention influences a parameter which has a beneficial effect on a disease or a condition. One such example is that modulation of LXR activity leads to decreased levels of serum cholesterol or certain lipoproteins which in turn have a beneficial effect on the prevention and treatment of atherosclerosis. Herein a condition is a physiological or phenotypic state which is desirably altered. One such example would be obesity which is not necessarily medically harmful but nonetheless a non desirable, phenotypic condition. In a preferred embodiment of the invention the method for prevention or treatment of a LXR receptor protein mediated disease or condition is applied to a human. This may be male or female.

Pharmaceutical compositions generally are administered in an amount effective for treatment or prophylaxis of a specific condition or conditions. Initial dosing in human is accompanied by clinical monitoring of symptoms, such symptoms for the selected condition. In general, the compositions are administered in an amount of active agent of at least about 100 ug/kg body weight. In most cases they will be administered in one or more doses in an amount not in excess of about 20 mg/kg body weight per day. Preferably, in most cases, doses is from about 100 ug/kg to about 5 mg/kg body weight, daily.

For administration particularly to mammals, and particularly humans, it is expected that the daily dosage level of active agent will be 0.1 mg/kg to 10 mg/kg and typically around 1 mg/kg.

By “therapeutically effective amount” is meant a symptom-alleviating or symptom-reducing amount, a cholesterol-reducing amount, a cholesterol absorption blocking amount, a protein and/or carbohydrate digestion-blocking amount and/or a de novo cholesterol biosynthesis-blocking amount of a compound according to the invention.

Likewise, the invention concerns a method of treating in mammal a disease which is correlated with abnormal cholesterol, triglyceride, or bile acid levels or deposits comprising administering to a mammal in need of such treatment a therapeutically effective amount of a compound according to the invention.

Accordingly, the compounds according to the invention may also be used as a method of prevention or treatment of mammalian atherosclerosis, gallstone disease, lipid disorders, Alzheimer's disease, skin disorders, obesity or cardiovascular disorders such as coronary heart disease or stroke.

The invention further concerns a method of blocking in a mammal the cholesterol absorption in the intestine in need of such blocking comprising administering to a mammal in need of such treatment a therapeutically effective amount of a compound according to the invention. The invention may also be used to treat obesity in humans.

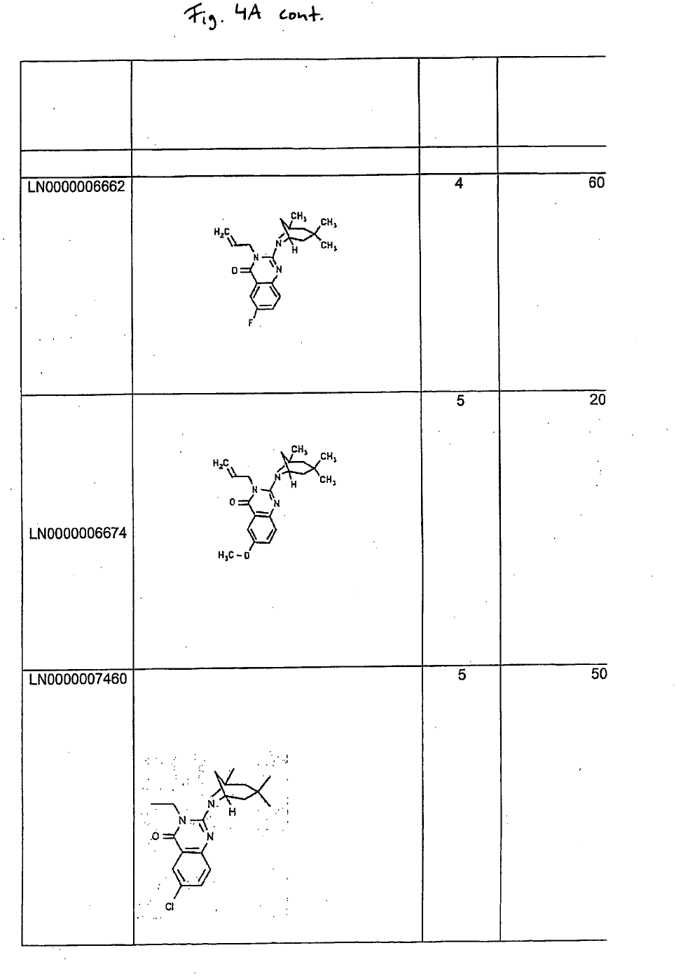

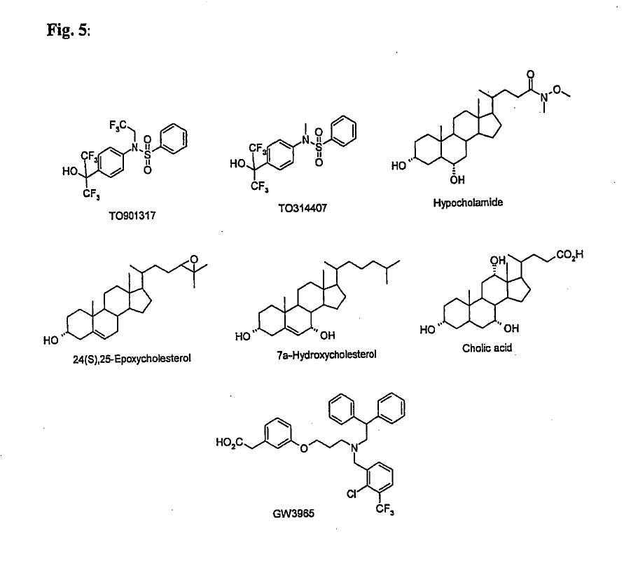

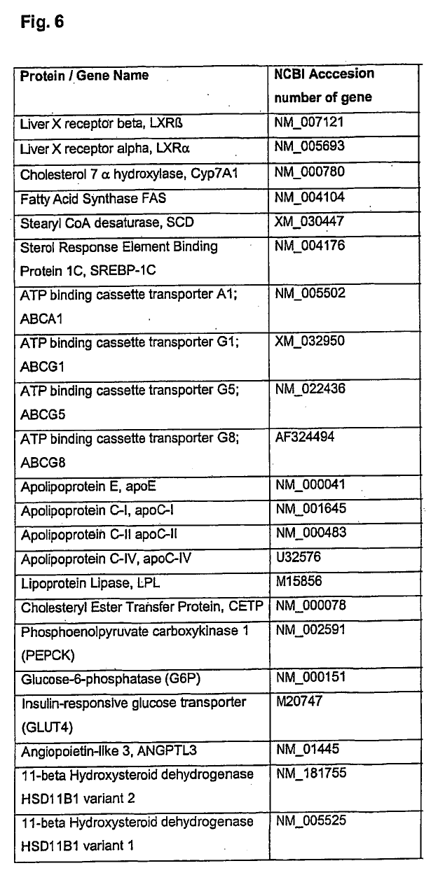

The Liver X Receptor alpha is a prototypical type 2 nuclear receptor meaning that it activates genes upon binding to the promoter region of target genes in a heterodimeric fashion with Retinoid X Receptor. The relevant physiological ligands of LXR are oxysterols The present compounds according to the invention have been demonstrated to have a high binding efficacy (binding coefficients measured as EC₅0 in the range 100 nM to 1500 nM) as well as agonistic and/or antagonistic properties. Consequently they may be applied to regulate genes that participate in bile acid, cholesterol and fatty acid homeostasis as well as other down-stream regulated genes. Examples of such genes are but are not limited to lipid absorption, cholesterol biosynthesis, cholesterol transport or binding, bile acid transport or binding, proteolysis, amino acid metabolism, glucose biosynthesis, protein translation, electron transport, and hepatic fatty acid metabolism. LXR often functions in vivo as a heterodimer with the Retinoid X Receptor. Published LXR agonists such as the “Tularik” compound “T0901317” (See FIG. 5) are known to influence the regulation of various liver genes. Genes found to be regulated by T0901317 can be found in FIG. 6. Thus, the invention also concerns a method of modulating a gene whose expression is regulated by the LXR receptor in a mammal comprising administration of a therapeutically effective amount of a compound according to the invention to said mammal.

A number of direct and indirect LXR target genes have been described whose regulated expression contribute to cholesterol homeostasis and lipogenesis. In this respect the direct regulation of Cyp7A, which was shown to be a direct target gene of LXR at least in the rodent lineage is an important aspect of cholesterol removal by increased metabolism of bile acids (Lehmann et al., J Biol. Chem. 272 (6) 3137-3140; 1007). Gupta et al. (Biochem. Biophys Res. Corn, 293; 338-343, 2002) showed that LXR a regulation of Cyp7A is dominant over FXR inhibitory effects on Cyp7A transcription.

A key transcription factor that was also shown to be a direct target gene for the LXR receptor is SREBP-1C (Repa et al., Genes and Development, 14: 2819-2830; 2000: Yoshikawa et al.; Mol. Cell. Biol. 21 (9) 2991-3000,2001).SREBP-1C itself activates transcription of genes involved in cholesterol and fatty acid synthesis in liver but also other mammalian tissues. Some of the SREBP-1C target genes involved in lipogenesis like FAS and SCD have shown to be additionally direct targets of the LXR receptors (Joseph et al.; J Biol Chem. Mar. 29, 2002; 277 (13): 11019-25; Liang et al., J Biol Chem. Mar. 15, 2002; 277 (11): 9520-8.).

A primary limitation for the applicability of LXR agonists as e. g. anti-atherosclerotic drugs comes from the observation that compounds with full agonistic activity, e.g. T0901317, not only elevate HDL cholesterol levels but do also increase plasma triglyceride levels in mice (Schultz et al., 2000 Genes Dev. 14: 2831-8.).

Concomitantly, not only genes that are involved in cholesterol efflux such as the cholesterol transporter ABCA1 (Venkateswaran et al., 2000 PNAS. 97: 12097-102.), ABCG1 as well as the lipid binding protein Apoliprotein E (Laffite et al. 2001 PNAS 98: 507-512) are induced by full LXR agonists, but also genes involved in lipogenesis, including the fatty acid synthase FAS (Joseph et al 2002 J Biol Chem. 277: 11019-11025), and SREBP-1c (Yoshikawa et al., 2001 Mol Cell Biol. 21: 2991-3000). Elevation of serum triglyceride levels is an independent risk factor for atherosclerosis (for review see Miller (1999) Hosp Pract (Off Ed) 34: 67-73.). Thus, LXR activity needs to be selectively modulated for therapeutic benefit. In particular, compounds need to be found that stimulate reverse cholesterol transport, but do not significantly increase trigclyceride levels.

Another gene that has been shown to be directly regulated by LXRs is the LPL gene, that codes for a key enzyme that is responsible for the hydrolysis of triglycerides in circulating lipoprotein, releasing free fatty acids to peripheral tissues. (Zhang et al. J Biol Chem. Nov. 16, 2001; 276 (46): 43018-24. ) This enzyme is believed to promote uptake of HDL cholesterol in liver, thereby promoting reverse cholesterol transport. A similar functional involvement in HDL clearance is described for the CETP gene product that facilitated the transfer of HDL cholesterol esters from plasma to the liver. LXR response elements were found in the CETP promoter and direct activation of this gene by LXR was demonstrated (Luo and Tall; J Clin Invest. February 2000; 105 (4): 513-20.).

The regulated transport of cholesterol through biological membranes is an important mechanism in order to maintain cholesterol homeostasis. A pivotal role in these processes in multiple tissues like e. g. macrophages and intestinal mucosa cells is maintained by the ATP-binding cassette transporter proteins (ABC). ABCA1 and ABCG1 were identified as direct LXR target genes (Costet et al.; J Biol Chem. September 8, 2000; 275 (36): 28240-5) that mediate cholesterol efflux and prevent thereby e.g. generation of artherogenic plaques in macrophages (Singaraja et al. J Clin Invest. July 2002; 110 (1): 35-42). Other ABC transporters like ABCG5 and ABCG8, primarily expressed in hepatocytes and enterocytes have also been reported to be directly responsive to LXR agonists (Repa et al., J Biol Chem. May 24, 2002; 277 (21): 18793-800. Kennedy et al., J Biol Chem. Oct. 19, 2001; 276 (42): 39438-47) and mediate the secretion of sterols from the liver and efflux of dietary sterols from the gut.

Apolipoproteins E, C-1, C-II, and C-IV, that fulfill important roles in lipoprotein/lipid homeostasis have also been shown to be direct targets of the LXR receptor (Laffitte et al., Proc Natl Acad Sci USA. Jan. 16, 2001; 98 (2): 507-12; Mak et al.; J Biol Chem. May 24, 2002 [epub ahead of print]). These proteins have been found to be crucial components of chylomicrons, VLDL, IDL, and HDL and are among other things associated with hypertriglyceridemia and arteriosclerosis.

Recently the LXRa itself was shown to be regulated by both LXR receptors in human cell types including macrophages suggesting an autoregulatory amplification event in the response to LXR ligands which could e. g. lead to an enhanced stimulation of LXR target genes like e. g. ABCA1 (Bolten et al.; Mol Endocrinol. 2002 March 2002; 16,(3): 506-14.; Laffitte et al., Mol Cell Biol. November 2001; 21 (22): 7558-68; Whitney et al.; J Biol Chem. Nov. 23, 2001; 276 (47): 43509-15).

Besides the important function of LXR receptors in tissues like liver and macrophages it has recently been reported that that stimulation of epidermal differentiation is mediated by Liver X receptors in murine epidermis. Differentiation maker genes like involucrin, loricin and profilaggrin have been shown to be upregulated upon LXR ligand treatment (Komuves et al.; J Invest Dermatol. January 2002; 118 (1): 25-34.).

Another recent report describes the regulation of cholesterol homeostasis (primarily the regulation of ABCA1, ABCG1, and SREBP-1C) by the LXR receptors in the central nervous system suggesting that LXRs may prove beneficial in the treatment of CNS diseases such as Alzheimer's and Niemann-Pick disease that are known to be accompanied by dysregulation of cholesterol balance (Whitney et al.; Mol Endocrinol. June 2002; 16 (6): 1378-85).

Activation of LXR by an agonist improves glucose tolerance in a murine model of diet-induced obesity and insulin resistance. Gene expression analysis in LXR agonist-treated mice reveals coordinate regulation of genes involved in glucose metabolism in liver and adipose tissue, e.g. the down-regulation of peroxisome proliferator-activated receptor gammacoactivator-1 alpha (PGC-1), phosphoenolpyruvate carboxykinase (PEPCK), and glucose-6-phosphatase expression and induction of glucokinase in liver. In adipose tissue, activation of LXR led to the transcriptional induction of the insulin-sensitive glucose transporter, GLUT4. LXR agonist may limit hepatic glucose output and improve peripheral glucose uptake (Laffitte et al. (2003) PNAS 100: 5419-24).

Therefore one other important embodiment of the invention concerns methods that enhances or suppresses amongst other today yet unknown LXR target genes the above mentioned genes and the associated biological processes and pathways through LXR compounds that are subject of this invention.

The compounds according to the invention may be used as medicaments, in particular for the manufacture of a medicament for the prevention or treatment of a LXR receptor protein or LXR receptor protein homologue mediated disease or condition in a mammal wherein the prevention or treatment is directly or indirectly accomplished through the binding of the compound according to the invention to the LXR receptor protein or LXR receptor protein homologue. These pharmaceutical compositions contain 0.1% to 99.5% of the compound according to the invention, more particularly 0.5% to 90% of the compound according to the invention in combination with a pharmaceutically acceptable carrier.

The invention concerns also the use of a compound according to the invention for the manufacture of a medicament for the prevention or treatment of a LXR receptor protein mediated disease or condition wherein the mammal described above is a human. The medicament may be used for regulating the cholesterol transport system, for regulating levels of cholesterol, triglyceride, and/or bile acid in a mammal preferentially a human by activating the LXR receptor. The medicament may be used for the treatment of atherosclerosis, gallstone disease, lipid disorders, Alzheimer's disease, skin disorders, obesity or a cardiovascular disorder.

The invention further concerns the use of a compound according to the invention for the manufacture of a medicament capable for blocking in a mammal, preferentially a human the cholesterol absorption in the intestine. Further the claimed compound may be used for the manufacture of a medicament for treating obesity in humans and for modulating a gene whose expression is regulated by the LXR receptor (see details above and figures).

The present invention shall now be further illustrated based on the following examples without being limited thereto. In the accompanying sequence protocol and the figures:

SEQ ID NO. 1 shows the protein sequence of the LRX alpha protein a portion of which was used for cloning as described in the examples,

SEQ ID NO. 2 shows the mRNA sequence of the LRX alpha protein,

SEQ ID NO. 3 shows the protein sequence of TIF2 (Acc. No: XM_{—}011633 RefSeq DB),

SEQ ID NO. 4 shows the respective mRNA sequence corresponding to the TIF2 protein,

SEQ ID NO 5 shows the protein sequence of the LXR beta protein a portion of which was used for cloning as described in examples,

SEQ ID NO 6 shows the mRNA sequence of the LXR beta protein,

SEQ ID NO 7 shows the sequence of primer (a) used in Example 1

SEQ ID NO 8 shows the sequence of primer (b) used in Example 1.

SEQ ID NO 9 shows the sequence of the response element used in Example 3.

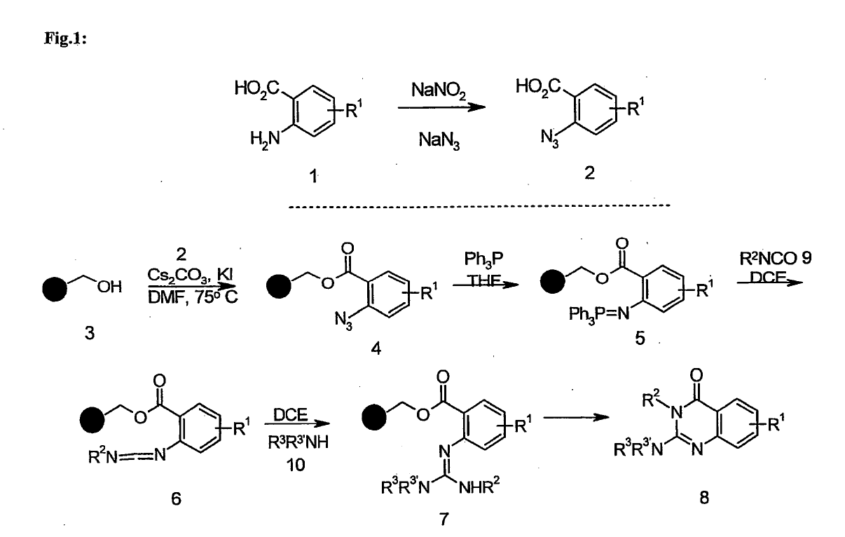

FIG. 1 shows the synthesis of the compounds according to the invention as also described in Example 2.

FIG. 2 shows the measurement parameters employed by the Wallace VICTOR2V™ Multilabel Counter which was used for measuring the EC₅₀ values (see also EXAMPLE 1)

FIG. 3 shows a table with the accession numbers for the key genes; FIG. 3A shows SEQ ID NO. 1 which is the protein sequence of the LRX alpha protein a portion of which was used for cloning as described in the examples. FIG. 3B shows SEQ ID NO. 2 which is the mRNA sequence of the LRX alpha protein. FIG. 3C shows SEQ ID NO. 3 which is the protein sequence of TIF2 (Acc. No: XM011633 Ref Seq DB), FIG. 3D shows SEQ ID NO. 4 which is the respective mRNA sequence corresponding to the TIF2 protein. FIG. 3E shows SEQ ID NO 5 which is the protein sequence of the LXR beta protein a portion of which was used for cloning as described in examples. FIG. 3F shows SEQ ID NO 6 which is the mRNA sequence of the LXR beta protein.

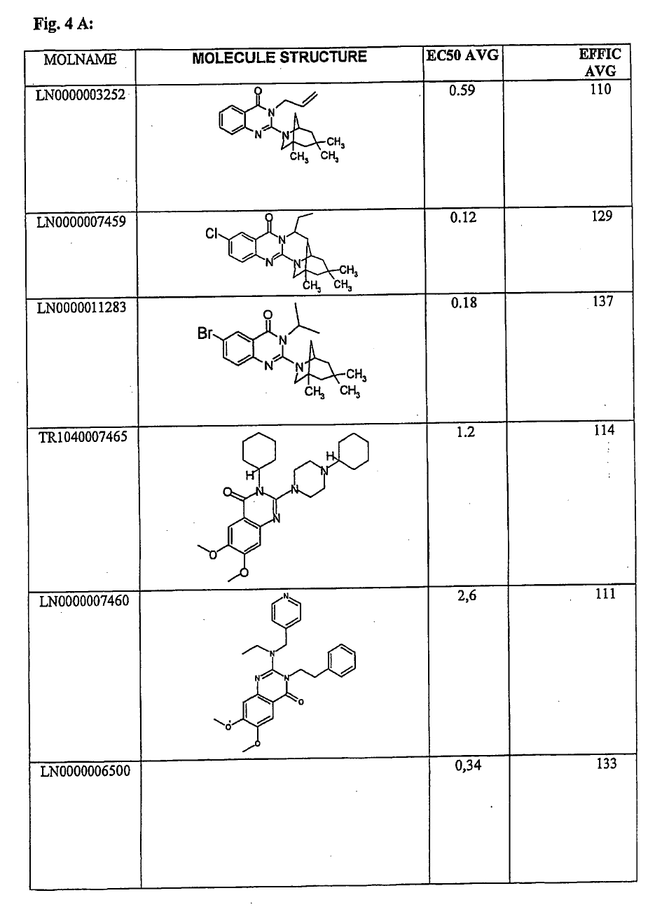

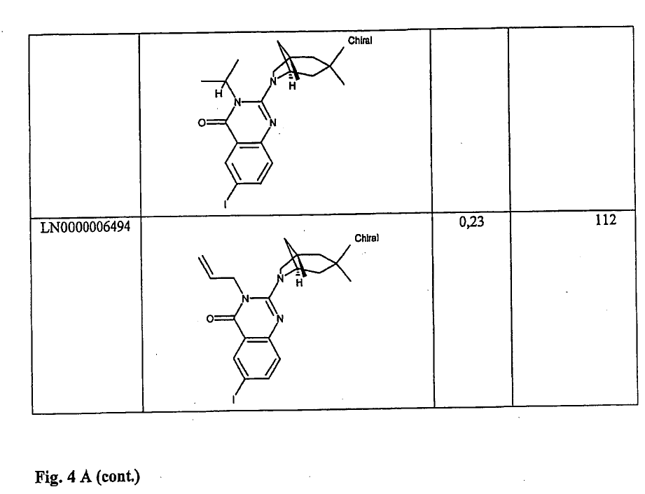

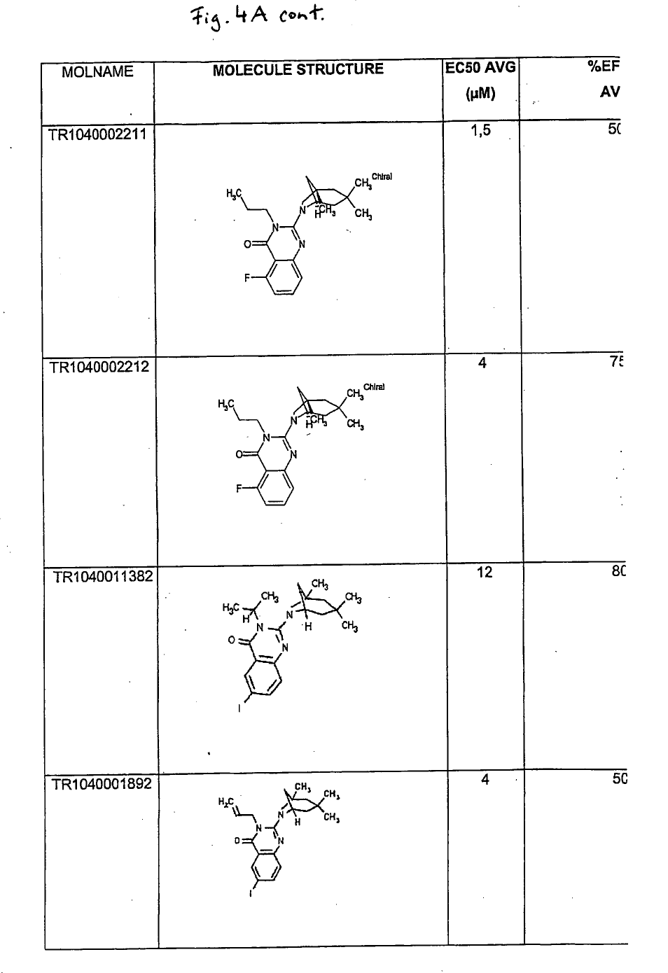

FIG. 4 shows the internal molecular name used by the applicant (MOLNAME) as well as the corresponding structures of preferred compounds according to the invention. The figure further shows their respective EC₅₀ values (EC₅₀ AVG) as established according to the experiment 1 in multiple experiments (see above), as well as their respective average efficacy (% activity relative to 22-(R)-hydroxycholesterol T0901317 control agonist).

FIG. 5 shows various known LXR ligands. The compound T0901317 is used as a reference compound here. It is apparent from their structures that the inventors have identified novel compounds which are structurally not related to these known ligands.

FIG. 6 shows various genes that have been found to be regulated through binding of an LXR agonist to the LXR protein.

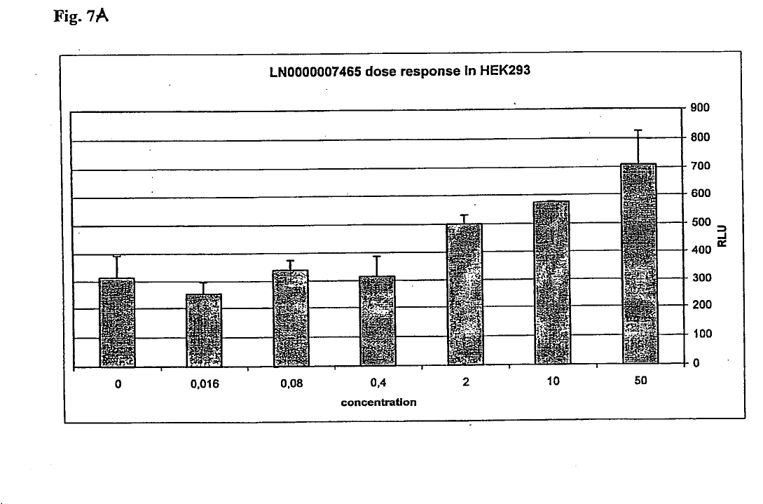

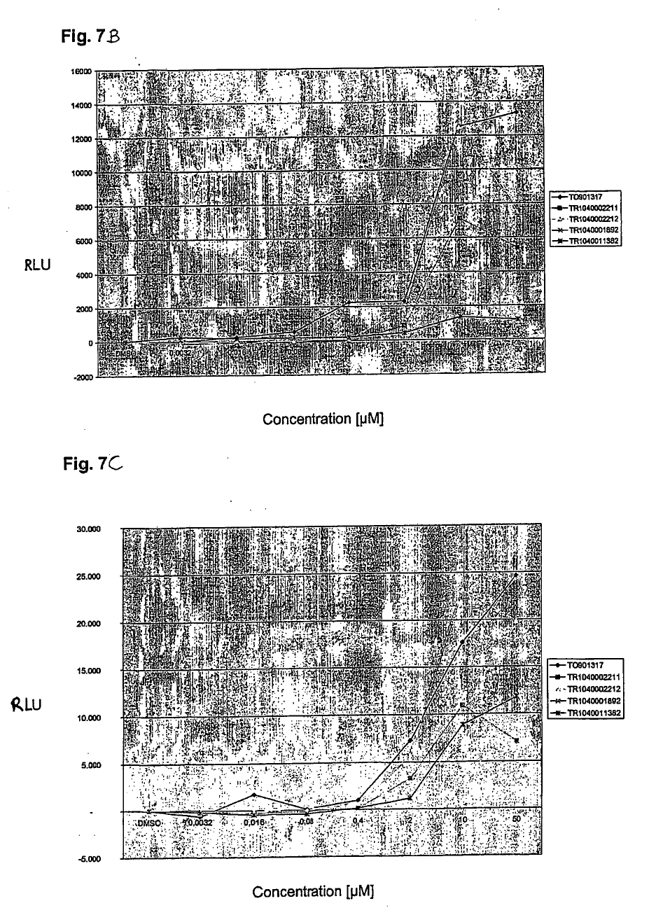

FIG. 7A shows a dose-dependent transactivation (EC₅₀˜3 uM) by LN0000007465 of the luciferase reporter gene via LXR alpha. FIG. 7B and FIG. 7C show dose dependence of inidcated compounds with LXR alpha LBD (7B) or LXR beta (7C) LBD containing constructs in mammalian one hybrid (M1 H) type assays. The respective pM concentrations of the compounds T0901317, TR1040002211, TR1040002212. TR1040001892 and TR1040011382 are given on the x-axis and the relative light units (RLU) are depicted on the y-axis.

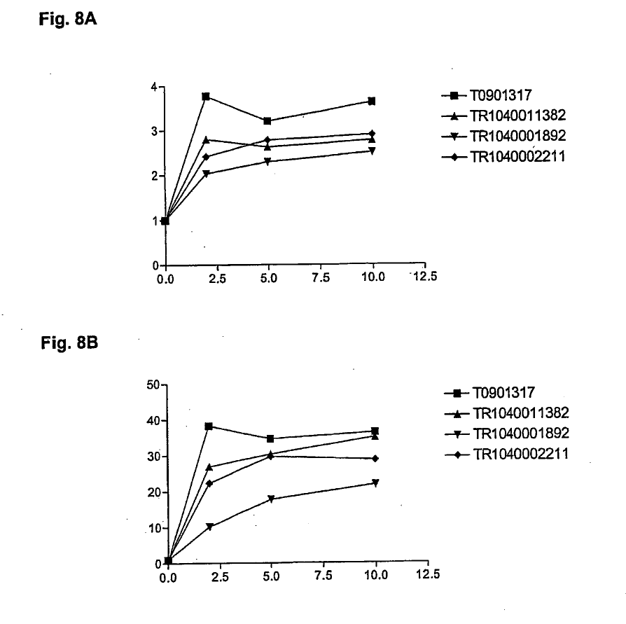

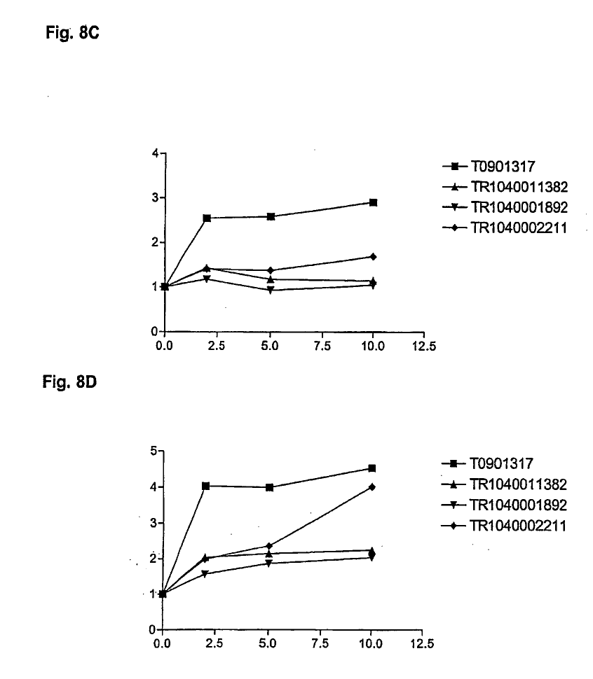

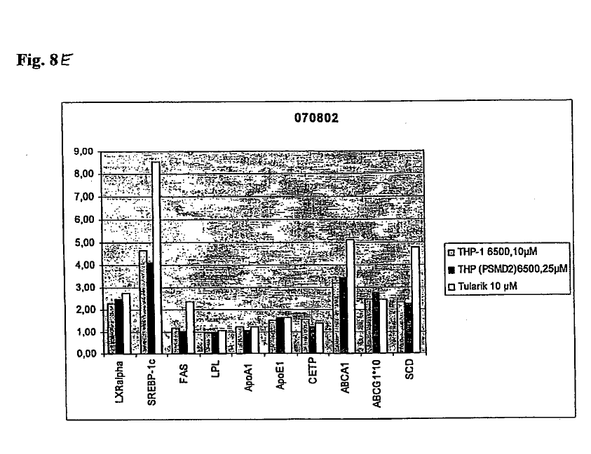

FIG. 8 shows the analysis of mRNA content of the indicated genes (ABCA1, ABCG1, FAS and SREBP-1 c) in total RNA isolated from THP-1 cells (8A and 8B) or HepG2 cells (8C and 8D) treated for 12 or 24 hours with indicated concentrations (pM on x-axis) of T0901317,TR1040011382, TR1040001892 and TR1040002211. The relative fold induction is depicted on the y-axis. (E) Analysis of mRNA content of the indicated genes in total RNA isolated from THP-1 cells treated for 24 hours with 2, 10 or 25 uM of LN0000006500 or 10 uM of the Tularik compound (T0901317). (F) Analysis of mRNA content of the indicated genes in total RNA from HepG2 cells treated for 24 hours with 2, 10 or 25 uM of LN0000006500 or 10 uM of the Tularik compound (T0901317).

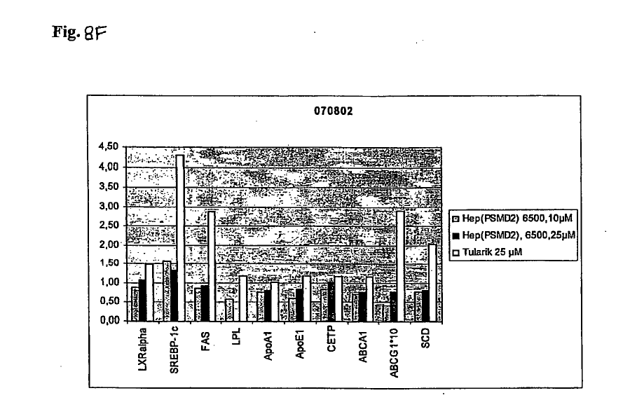

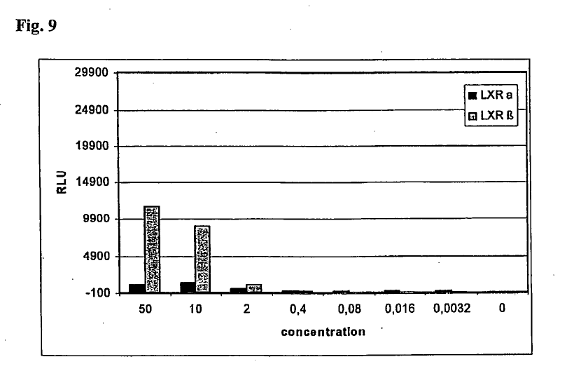

FIG. 9 shows the dose dependent transactivation by LN0000006500 of the pFR-luc reporter gene in CHO cells via Gal4 LBD-fusion constructs derived from LXRa or LXRb. Concentrations of the compound administered (uM) and RLLT's determined from extracts of cells are indicated.

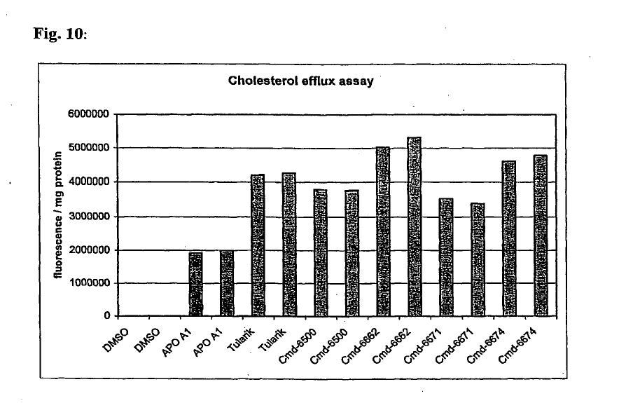

FIG. 10 shows the analysis of total cholesterol from supernatants of cultivated THP-=b 1 cells incubated without or with ApoA1 and ApoA1 plus 10 uM of the compounds Tularik (T0901317) or LN0000006500, LN0000006662, LN0000006671 or LN0000006672 as indicated.

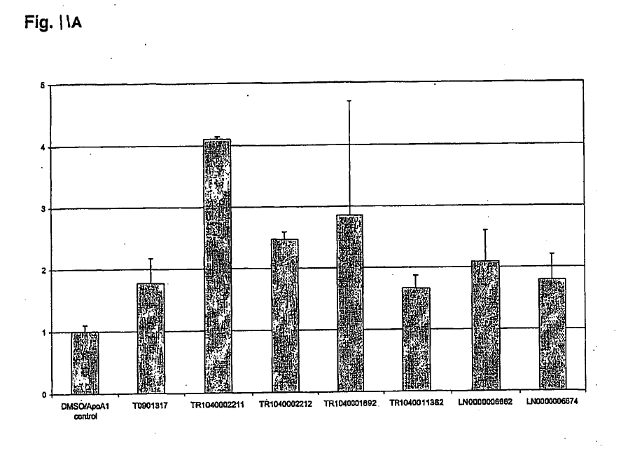

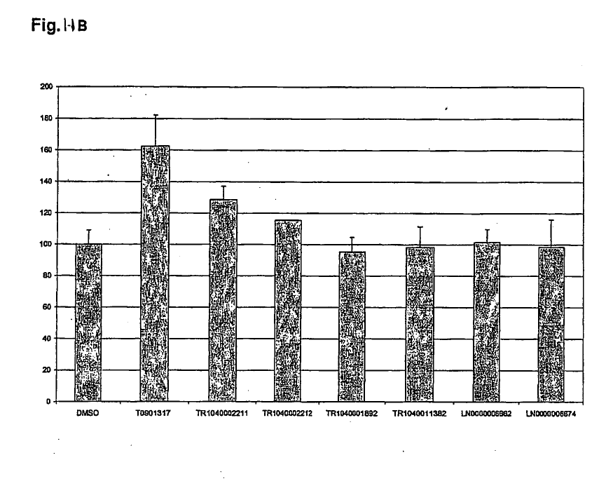

FIG. 11 shows an analysis of relative fold increase in total cholesterol from supernatants of cultivated THP-1 cells (indicated on the y-axis) incubated with ApoA1 and with or without 10 uM of the compounds T0901317, TR1040002211, TR1040002212. TR1040001892, TR1040011382, LN0000006662 and LN0000006674 as indicated on the X-axis of FIG. 11A. (B) Analysis of relative levels of total triglyceride (TG) content in HepG2 cells (indicated on the y-axis) treated with 25 pM of the indicated compounds T0901317, TR1040002211, TR1040002212. TR1040001892, TR1040011382, LN0000006662, and LN0000006674 (indicated on the x-axis).

### EXAMPLES

### Example 1

### In vitro Screening for Compounds which Influence LXR Binding to Coactivators

For screening purposes a GST and 6× His fusion of the LBD (from amino acids 155 of hLXRalpha to 447) of human LXRalpha was constructed by first cloning a Gateway cassette (Invitrogen) in frame into the Sma I site of the pAGGHLT Polylinker (Pharmingen). Then a PCR fragment specifically amplified from human liver cDNA was cloned into the resulting pACGHLT-GW following the manufacturers instructions for Gateway cloning (Invitrogen) to yield pACGHLT-GW-hLXRalphaLBD.

Primers used for amplification were:

(SEQ ID NO 7)

primer (a)
GGGGACAAGTTTGTACAAAAAAGCAGGCTCGCTTCGCAAATGCCGTCAG,

(SEQ ID NO 8)

and primer (b)
GGGGACCACTTTGTACAAGAAAGCTGGGTCCCCTTCTCAGTCTGTTCCAC
TT.

100% sequence integrity of all recombinant products was verified by sequencing. Recombinant Baculovirus was constructed from pACGHLT-GW-hLXRalphaLBD using the Pharmingen Baculovirus Expression vector system according to instructions of the manufacturer. Monolayer cultures of SF9 cells were infected by the virus as recommended by Pharmingen or 200 ml cultures of 1×10⁶ cells/ml grown in 2 liter Erlenmeyer flasks on an orbital shaker at 30 rpm were infected by 10 ml of same virus stock. In both cases cells were harvested 3 days after infection. All cell growth was performed in Gibco SF900 II with Glutamine (Invitrogen) medium without serum supplementation at 28° C. Since SF9 cells contain significant amounts of endogenous 30 GST, purification was performed via His and not via GST affinity chromatography.

To this end instructions of Pharmingen for purification of recombinant His tagged proteins from SF9 cells were followed with the following modifications: All detergents were omitted from the buffers and cells were lysed on ice by 5 subsequent sonication pulses using a sonicator needle at maximum power.

All eluates were dialyzed against 20 mM Tris/HCl pH 6.8, 300 mM KCl; 5 mM MgCl₂;1 mM DTT; 0.2 mM PMSF; 10% Glycerol. A typical dialyzed eluate fraction contained the fusion protein at a purity of more than 80%. Total protein concentration was 0.1-0.3 mg/ml.

For E. coli expression of a NR coactivator, pDest17-hTif2BD expressing an NR interaction domain from amino acids 548-878 of human Tif2 (Acc. No: XM011633 Ref Seq) tagged by 6 N-terminal His residues was constructed. Therefore, a PCR fragment specifically amplified from human liver cDNA was subcloned into pDest 17 (Invitrogen) following the manufacturers instructions for Gateway cloning (Invitrogen). Primers used for Amplification were:

primer (a)
GGGGACAAGTTTGTACAAAAAAGCAGGCTCGTTAGGGTCATCGTTGGCTT
CACC
and
primer (b)
GGGGACCACTTTGTACAAGAAAGCTGGGTCTCAAAGTTGCCCTGGTCGTG
GGTTA

For E. coli expression plasmid DNA was transformed into chemically competent E. coli BL21 (Invitrogen, USA) and cells were grown to an OD₆₀₀ of 0.4-0.7 before expression was induced by addition of 0.5 mM IPTG according instructions of the manufacturer (Invitrogen). After induction for 8 hours at 30° C. cells were harvested by centrifugation for 10 minutes at 5000×g. Fusion proteins were affinity purified using Ni—NTA Agarose (QIAGEN) according to the instructions of the manufacturer.

Recombinant Tif2 construct was dialyzed against 20 mM Tris/HCL pH 7.9; 60 mM KCl; 5 mM MgCl₂; 1 mM DTT, 0.2 mM PMSF; 10% glycerol. A typical dialyzed eluate fraction contained the fusion protein at a purity of more than 80%. Total protein concentration was 0.1-0.3 mg/ml.

The TIF2 fragment was subsequently biotinylated by addition of 5-40 ul/ml Tif2 fraction of a Biotinamidocaproate N-Hydroxysuccinimide-ester (Sigma) solution (20 mg/ml in DMSO). Overhead rotating samples were incubated for 2 hours at room temperature. Unincorporated label was then separated using G25 Gel filtration chromatography (Pharmacia Biotech, Sweden). Protein containing fractions from the column were pooled and tested for activity in the assay as described below.

For screening of compound libraries as provided for by the methods shown below in the examples for substances which influence the LXR/Tif 2 interaction, the PerkinElmer LANCE technology is applied. This method relies on the binding dependent energy transfer from a donor to an acceptor fluorophore attached to the binding partners of interest. For ease of handling and reduction of background from compound fluorescence LANCE technology makes use of generic fluorophore labels and time resolved detection (for detailed description see Hemmila I, Blomberg K and Hurskainen P, Time-resolved resonance energy transfer (TR-FRET) principle in LANCE, Abstract of Papers Presented at the 3 rd Annual Conference of the Society for Biomolecular Screening, Sep., California (1997)) For screening, 20-200 ng of biotinylated Tif 2 fragment and 10-200 ng of GST-LXR fragment are combined with 0.5-2 nM LANCEEu-(W1024) labelled anti-GST antibody (Perkin Elmer) and 0.1-0.5 pg of highly fluorescent APC-labelled streptavidin (Perkin Elmer, AD0059) in the presence of 50 pM of individual compounds to be screened in a total volume of 25 ul of 20 mM Tris/HCI pH 6.8; 300 mM KCl; 5 mM MgCl₂; 100-1000 ng/ul/BSA DMSO content of the samples is kept below 4%. Samples are incubated for a minimum of 60 minutes in the dark at room temperature in FIA-Plates black 384 well med. binding (Greiner).

The LANCE signal was detected by a Perkin Elmer VICTOR2V™ Multilabel Counter applying the detection parameters listed in FIG. 2. The results were visualized by plotting the ratio between the emitted light at 665 nm and at 615 nm. For every batch of recombinant proteins amount of proteins, including BSA and labeling reagents giving the most sensitive detection of hits was determined individually by analysis of dose response curves for 22R Hydroxycholesterol and T0901317

### Example 2

### Experimental Procedure for the Preparation of the Compounds According to the Invention

### O-AZIDOBENZOIC ACID SYNTHESIS (2)

The anthranilic acid (1, 1 eq., 0.5-1 M) was suspended in 6 M HCl, containing enough AcOH (0-20% dependent upon the anthranilic acid) to facilitate dissolution of the anthranilic acid and/or the intermediate diazonium salt, and cooled to 0° C. NaNO₂ (1.1 eq., 1.3-2.5 M) dissolved in H₂O was added to the anthranilic acid solution at a rate such that the temperature of the reaction solution remained below 5° C. The resulting homogeneous solution of the diazonium salt was slowly filtered through a sintered glass funnel into a solution of NaN₃ (1.1 eq., 0.7-1.1 M) and NaOAc (12 eq.) in H₂O. The reaction mixture was stirred/shaken for 30-60 min following cessation of vigorous N₂ evolution. Following acidification of the reaction mixture to pH 1 with concentrated HCI, the mixture was cooled to 0° C. to encourage complete precipitation of the o-azidobenzoic acid. The precipitate is collected by filtration and washed with 6 M HCl (2×) and H₂O (2×). The o-azidobenzoic acid product (2) is dried in vacuo (500 mtorr, 30° C.).

### ACYLATION OF HYDROXYMETHYL RESIN (4)

To hydroxymethyl resin (1.0 eq., 1.3 mmol/g) and the o-azidobenzoic acid (1, 2.5 eq.) is added DMF (to give 400 mM o-azidobenzoic acid, 1), CsCO₃ (2.0 eq.) and K1 (2.0 eq.). Following agitation of the reaction mixture for 36-48 h, the resin-bound o-azidobenzoic acid (4) is washed with MeOH (2 cycles), CH₂Cl₂ (3 cycles), MeOH (3 cycles), DMF (3 cycles), MeOH (3 cycles) and CH₂Cl₂ (3 cycles), and dried in vacuo.

### AZA-WITTIG FORMATION (5)

To the resin-bound o-azidobenzoic acid (4, 1.0 eq.) was added a solution of PPh3 (THF, 500 mM, 5.0 eq.). After 6 h, the resin was washed with 3 cycles of the following: THF (3 cycles), toluene (3 cycles), CH₂Cl₂ (3 cycles) and hexanes (3 cycles). Followed by drying in vacuo to afford resin bound iminophosphorane (5)

### CARBODIIMIDE FORMATION (6)

To the resin-bound iminophosphorane (5, 1 eq.) was added isocyanate (9, 5 eq., 450 mM) dissolved in ClCH₂CH₂Cl. The compounds were shaken at ambient temperature for 16 h, washed with 3 cycles of the following: THF (3 cycles), toluene (3 cycles), CH₂Cl₂ (3 cycles) and hexanes (3 cycles), and dried in vacuo to afford carbodiimide (6).

### GUANIDINE FORMATION/CYCLIZATION

To the carbodiimide functionalized resin (6) was added secondary amine (10, 0.6 eq., 500 mM) dissolved in ClCH₂CH₂Cl. The reaction mixture was heated to 50° C. in an incubator for 12-72 h to afford 2-aminoquinazoline (8).

All of the final products were analyzed by HPLC using mass and an Evaporative Light Scattering Detector (ELSD) detection to determine purity and identity.

### Example 3

This example illustrates that a compound according to the invention (experiments shown were done with MOLSTRUCTURE) can mediate transactivation of LXR mediated transcription in HEK293 cells.

This example illustrates that compounds according to the invention (experiments shown were done with MOLSTRUCTURE LN 0000007465 (see FIG. 4 for structural formula), TR1040001892, TR1040011382, TR1040002211, and TR1040002212 (see formulas (9) to (12) for structural formulas)) activate luciferase reporter gene expression in a dose dependent manner mediated through GAL4-LXRa-LBD or GAL4-LXRb-LBD constructs in HEK293 cells.

TR1040001892 and TR1040011382 do activate LXR beta LBD truct mediated luciferase activity much stronger than with LXR alpha construct which is in contrast to the similar activation of both LXR alpha and LXR beta LBD containing constructs by TR1040002211 and T0901317.

For LN 0000007465, HEK293 cells were grown in 48 well plates and co-transfected with the pTRexDest30 (Invitrogen) derivatives pTRexDest30-hLXRa, pTRexDest30-hRXRO and the pGL2 promoter (Promega) derivative pGL2 promoter-LXRRE (each 300 ng of plasmid DNA). The full length human LXR (accession U68233) and the full length human RXRa (accession P19793) were cloned into the pTRexDest30 applying the manufacturer protocols for the Gateway system (Invitrogen). The LXR response elements (LXRRE) were 5′ CccttTGGTCActcaAGTTCAagtgatgatagaattcggatccttTGGTCActcaAGTTCAagtg A 3′ (SEQ ID NO. 9) derived from the rat Cyp7a promoter (Laffite et al., 2001, PNAS 98, pp 507).

Luciferase reporter activity was measured in triplicates from extracts of cells after incubating cells in culture medium (DMEM [Gibco-BRL]+10% FCS [PAA laboratories]) for 16 hours (5% CO₂, 37° C.) containing 0.5% DMSO (control) or 0.5% DMSO with increasing concentrations of LN0000007465. A dose-dependent transactivation (EC₅₀˜3 pM) of the reporter gene by LXRa was observed (FIG. 7A).

For TR1040001892, TR1040011382, TR1040002211, and TR1040002212, HEK293 cells are grown in 96 well plates and co-transfected with pFR-luc (Stratagene) and pCMV-BD-LXRa-LBD or pCMV-BD-LXRb-LBD (each 100 ng of plasmid DNA per well). Transfection is carried using Lipfectamine 2000 (Gibco-BRL) according to the manufacturers protocol. The ligand binding domains (LBD) of LXRa and LXRb are cloned into the pCMV-BD-GW (the Gateway Reading Frame Cassette B is cloned as an EcoRV fragment into Sma1 site of pCMV-BD) applying the manufacturer protocols for the Gateway system (Invitrogen).

Luciferase reporter activity is measured in triplicates from extracts of cells after incubating cells in culture medium (DMEM [Gibco-BRL]+10% FCS [PAA laboratories]) for 16 hours (5% CO₂, 37° C.) containing 0.5% DMSO (control) or 0.5% DMSO with increasing concentrations of TR1040001892, TR1040002211 or T0901317 (Sigma T 2320, see FIG. 5 for structural formula). The type of assay used here is a mammalian one hybrid (M1 H) assay that is known to those skilled in the art. Dose-dependent luciferase activities originating from pFR-luc demonstrate the relative activities of the compounds with the LXRa or LXRb LBDs in this mammalian one hybrid type approach.

### Example 4

This example shows that described compounds can increase the abundance of mRNA of target genes for the LXR proteins like ABCA1 and ABCG1 in THP-1 cells which are treated with TPA or FAS and SREBP-1c in HepG2 cells as shown in FIG. 8A-F.

THP-1 (3×10⁵ cells per dish) cells were seeded in 24 well dishes in 3 ml modified RPMI-1640 medium (ATTC, Cat. No. 30-2001) containing 10% FCS (GIBCO) and 100 nM TPA and cultivated at 37° C. in 5% CO₂ for 24 or 48 hours. HepG2 (4.5×10⁵ cells per dish) were seeded in 24 well dishes in 3 ml DMEM Medium containing 10% FCS (GIBCO) and cultivated at 37° C. in 5% CO₂ for 48 hours as indicated in FIG. 8B or until they are approx. 60% confluent.

The medium was then removed and replaced with medium containing 10% charcoal/stripped FCS (Hyclone) for 12 h. Treatment is done with LN0000006500 at 2, 10 or 25 uM concentration or Tularik (T0901317) at 10 uM for 24 hours as indicated in FIG. 8A as an example or for 12 h (THP-1 cells) and 24 h (HepG2 cells), respectively, in medium containing 10% charcoal/dextran-stripped FCS (and 100 nM PMA in the case of THP-1 cells).

LXR compounds are dissolved in DMSO, with the final solvent concentration never exceeding 0.125%. All treatments are done in triplicates and experiments repeated twice. Total RNA is extracted using the Qiagen Rneasy Mini Kit and treated with DNase (DNA free kit, Ambion). RNA is reverse transcribed with Oligo (dT) primer and real-time reverse transcription PCR (TaqMan) is performed using the ABI Prism 7900HT Sequence Detection System and reagents supplied by Applied Biosystems. mRNA steady state levels are normalised to H3 histone (H3F3A) expression levels.

The sequences of forward primers, reverse primers and TaqMan probes are as follows

FAS: ctgagacggaggccatatgct, gctgccacacgctcctctag,
FAM-cagcagttcacggacatggagcacaa-TAMRA
ABCA1: tcctgtggtgtttctggatgaac,
cttgacaacacttagggcacaattc,
FAM-accacaggcatggatcccaaagcc-TAMRA

The fold change of mRNA abundancy of compound LN0000006500 or Tularik treated versus DMSO treated as a control is shown in FIGS. 8E and F for several analyzed target genes indicated in FIGS. 8E and F.

### Example 5

This example shows that described compounds LN0000006500 or Tularik can selectively enhance transcription mediated by the LBD's of the respective nuclear receptors LXRa and LXRb.

CHO cells (1×10⁵ cells 96 well plate) were co-transfected (Lipofectamine 2000 GIBCO) with pFR-luc (Stratagene) as a reporter gene construct and pCMV-AD derivatives containing the LXRa or LXRb ligand binding domains, which were cloned via the gateway system (GIBCO) described in Example 1, in order to express Gal4DBD-LXRa or Gal4DBD-LXRb fusion proteins.

Cells were grown in DMEM containing 10% FCS at 37° C. in 5% humidified CO₂ for 16 h in presence of 0.05% DMSO vehicle or 0.032 to 50 uM LN0000006500 in vehicle (as indicated in FIG. 9). Luciferase activity was determined from aliquots of extracts prepared from cells following standard luciferase assay kits and protocols from Promega.

### Example 6

All compounds T0901317, TR1040002211, TR1040002212. TR1040001892, TR1040011382, LN0000006662, and LN0000006674 cause a marked increase in cholesterol export in differentiated THP-1 macrophages (see FIGS. 9 and 11).

Strikingly, the compound T0901317 causes a marked increase in triyglyceride mass in HepG2 liver cells, while compounds like TR1040001892 and TR1040011382 do not cause a significant increase in triglyceride mass. Compounds TR1040002211 and TR1040002212 cause a slight increase in triglyceride mass.

This behaviour is similar to the selective transcriptional effect of compounds like TR1040001892 and TR1040011382 on the LXR target genes in HepG2 versus THP-1 cells.

Methods: Cultures of the monocyte-macrophage cell line and the hepatocytes HepG2 are obtained from the American Type tissue Culture Collection, Rockville, Md. and were grown in RPMI 1640 medium supplemented with 10% fetal bovine serum, 10 mM HEPES, 2 mM pyruvate, 50 uM (3-Mercaptoethanol (THP-1) and Minimum essential medium (Eagle) with 2 mM L-glutamine and Earle's BSS supplemented with 10% fetal bovine serum, 2 mM glutamin, 0.1 mM non-essential amino acids, 1 mM sodium pyruvate (HepG2), respectively, at 37° C. in 5% CO₂.

THP-1 cells are differentiated into macrophages by addition of 100 nM Phorbol 12-Myristate 13-Acetate (PMA; Sigma P8139) and PMA included in the medium of all subsequent experiments to maintain differentiation.

For cholesterol efflux measurements and triglyceride analysis cells are seeded in 6 well plates at 1.8×10⁶ cells (THP-1) and 1×10⁶ cells (HepG2) per well, respectively.

CHOLESTEROL EFFLUX THP-1 cells are seeded in 6 well plates at 1×10⁶ cells per well in RPMI 1640 medium containing 10% FCS and 100 nM TPA for 72 h. After washing with PBS cells are incubated 24 h with fresh in RPMI 1640 medium containing 10% FCS and 100 nM TPA. Cells are washed twice with PBS and RPMI 1640 medium containing 0.15% BSA and 100 nM TPA is added for further 24 h. Treatment with LXR compounds is done for 24 h in RPMI 1640 medium containing 0.15% BSA, 100 nM TPA and 40 pg/ml ApoA1 (Calbiochem Kat. num; 178452). Medium is collected, centrifuged to remove cell debris and assayed for cholesterol using a commercial fluorometric kit (Molecular Probes A-12216). The remaining cellular proteins are lysed with 0.3 NNaOH and protein content measured with the Biorad Bradford reagent.

TRIGLYCERIDE ASSAY HepG2 cells are seeded in poly-L-Lysine coated 6 well plates at 1×10⁶ cells per well in EMEM medium containing 10% FCS until they were appr. 60% confluent. Before treatment with LXR compounds growth medium is changed to medium containing 10% charcoal/dextran-stripped FCS for 12 h. Treatment is done for 24 h in medium containing 10% charcoal/dextran-stripped FCS. Cells are washed twice with ice-cold PBS/0.2% BSA and twice with cold PBS and all liquid carefully removed. Triglyceride are extracted with 1.5 ml hexane/isopropanol=3: 2 per well with gentle shaking for 2-3 h at RT according to Pan et al. (2002) JBC₂77, 4413-4421 and Goti et al. (1998) Biochem J, 332, 57-65. The extraction solution is collected, dried under vacuum and redissolved in isopropanol/1.5% triton. The remaining cellular proteins are lysed with 0.3 N NaOH and protein content measured with the Biorad Bradford reagent.

Triglyceride levels are measured as esterified glycerol using a commercial enzymatic colorimetric kit (Sigma 343-25P). In a preliminary assay it is checked by omitting the lipase enzyme that contribution of free glycerol is negligible.

### Example 7

This example shows that described compounds at 10 ul concentration for 24 hours can increase the reverse cholesterol transport in THP-1 cells that were treated with TPA.

THP-1 (1×10⁶ cells per dish) cells were seeded in 6 well dishes in 3 ml modified RPMI 1640 medium (ATTC, Cat. No. 30-2001) containing 10% FCS (GIBCO) and 100 nM TPA and cultivated at 37° C. in 5% CO₂ for 72 hours. The medium was then removed and replaced with fresh medium containing 100 nM TPA and 0.15% BSA. After 24 h incubation the cells were washed in PBS and 1.5 ml of fresh medium containing either 0.1% DMSO alone or 0.1% DMSO together with 40 ug/ml ApoA1 (Calbiochem) or 40 pg/ml ApoA1 plus the in FIG. 10 as an example indicated compounds Tularik (T0901317), LN0000006500, LN0000006662, LN0000006671, LN0000006674 at 10 uM.

After incubation for 24 hours, total cholesterol was determined from cell supernatant in each of the wells using an enzymatic assay with fluorescence read-out for the determination of cholesterol (Amplex Red Cholesterol Assay Kit (A-12213). The fluorescence readout per mg of total protein content as determined from cells that were present in the respective well are shown in FIGS. 9 or 11 as an example.

1. A compound of the formula (1), or pharmaceutical acceptable salts or solvates thereof according to formula (1) 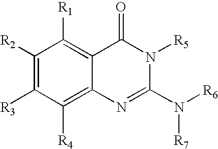
wherein:
R₁, R₂, R₃ and/or R₄, is independently from each other H, halogen, hydroxy, protected hydroxy, cyano, nitro, C₁ to C₆ alkyl, C₁ to C₆ substituted alkyl, C₁ to C₇ alkoxy, C₁ to C₇ substituted alkoxy, C₁ to C₇ acyl, C₁ to C₇ substituted acyl, C₁ to C₇ acyloxy, carboxy, protected carboxy, carboxymethyl, protected carboxymethyl, hydroxymethyl, protected hydroxymethyl, amino, protected amino, (monosubstituted)amino, protected (monosubstituted)amino, (disubstituted)amino, carboxamide, protected carboxamide, N—(C₁ to C₆ alkyl)carboxamide, protected N—(C₁ to C₆ alkyl)carboxamide, N,N-di(C₁ to C₆ alkyl)carboxamide, trifluoromethyl, N—((C₁ to C₆ alkyl)sulfonyl)amino, N-(phenylsulfonyl)amino or phenyl, wherein the phenyl is substituted or unsubstituted,
R₅ is H, C₁ to C₈ alkyl, C₁ to C₈ substituted alkyl, C₇ to C₁₂ alkylphenyl or C₇ to C₁₂ substituted phenylalkyl,
R₆ is H, C₁ to C₈ alkyl, C₁ to C₈ substituted alkyl, C₇ to C₁₂ alkylphenyl or C₇ to C₁₂ substituted phenylalkyl, and
R₇ is H, C₁ to C₈ alkyl, C₁ to C₈ substituted alkyl, C₇ to C₁₂ alkylphenyl or C₇ to C₁₂ substituted phenylalkyl.

2. The compound according to claim 1 wherein R₆ and R₇ are taken together with nitrogen to form a heterocycle or substituted heterocycle or a heteroaryl or substituted heteroaryl according to the following formula (2).

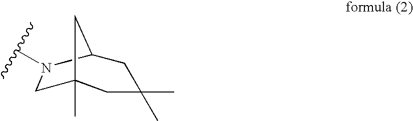

3. The compound according to claim 2, or pharmaceutical acceptable salts or solvates thereof, wherein:
R₁, R₂, R₃, R₄, is H, halogen, hydroxy, protected hydroxy, cyano, nitro, C₁ to C₆ alkyl, C₁ to C₆ substituted alkyl, C₁ to C₇ alkoxy, C₁ to C₇ substituted alkoxy, C_{1 l to C}₇ acyl, C₁ to C₇ substituted acyl, C₁ to C₇ acyloxy, carboxy, protected carboxy, carboxymethyl, protected carboxymethyl, hydroxymethyl, protected hydroxymethyl, amino, protected amino, (monosubstituted)amino, protected (monosubstituted)amino, (disubstituted)amino, carboxamide, protected carboxamide, N—(C₁ to C₆ alkyl)carboxamide, protected N—(C₁ to C₆ alkyl)carboxamide, N, N-di(C₁ to C₆ alkyl)carboxamide, trifluoromethyl, N—((C₁ to C₆ alkyl)sulfonyl)amino, N-(phenylsulfonyl)amino or phenyl, and
R₅ is H, C₁ to C₈ alkyl, C₁ to C₈ substituted alkyl.

4. The compound according to claim 1 wherein R₆ and R₇ are taken together with nitrogen to form the heterocycle according to the following formula (3)

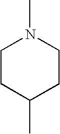

5. The compound according to claim 1 according to formula (Ia), 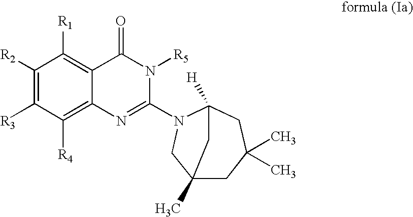
wherein R₁, R₂, R₃, R ₄ are independently from each other H, halogen, hydroxy, protected hydroxy, cyano, nitro, C₁ to C₆ alkyl, C₁ to C₆ substituted alkyl, C₁ to C₇ alkoxy, C₁ to C₇ substituted alkoxy, C₁ to C₇ acyl, C₁ to C₇ substituted acyl, C₁ to C₇ acyloxy, carboxy, protected carboxy, carboxymethyl, protected carboxymethyl, hydroxymethyl, protected hydroxymethyl, amino, protected amino, (monosubstituted)amino, protected (monosubstituted)amino, (disubstituted)amino, carboxamide, protected carboxamide, N—(C₁ to C₆ alkyl)carboxamide, protected N—(C₁ to C₆ alkyl)carboxamide, N,N-di(C₁ to C₆ alkyl)carboxamide, trifluoromethyl, N—((C₁ to C₆ alkyl)sulfonyl)amino, N-(phenylsulfonyl)amino or phenyl, and R₅ is H, C₁ to C₈ alkyl, C₁ to C₈ substituted alkyl, C₇ to C₁₂ alkylphenyl or C₇ to C₁₂ substituted phenylalkyl.

6. The compound according to claim 1 of the following formula (4)

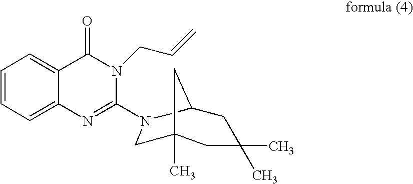

7. The compound according to claim 1 of the following formula (5)

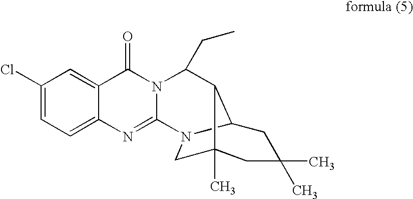

8. The compound according to claim 1 of the following formula (6)

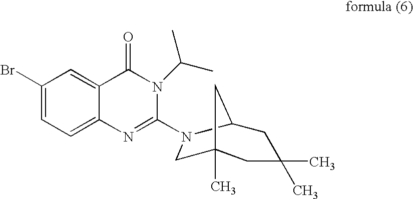

9. The compound according to claim 1 wherein R₆ and R₇ are taken together with nitrogen to form the heterocycle according to the following formula (7)

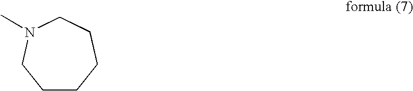

10. The compound according to claim 1 according to the following formula (8)

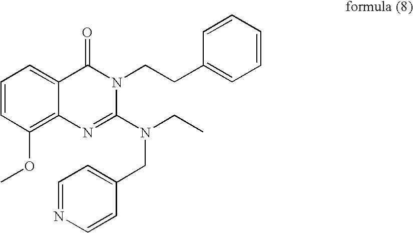

11. The compound according to claim 1 according to the following formula (8a)

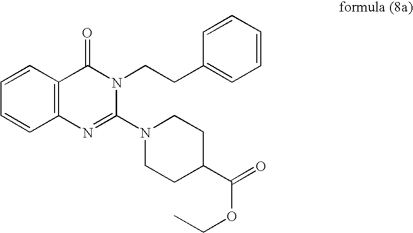

12. The compound according to claim 5 according to the following formula (9)

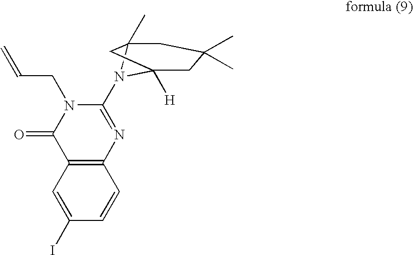

13. The compound according to claim 5 according to the following formula (10)

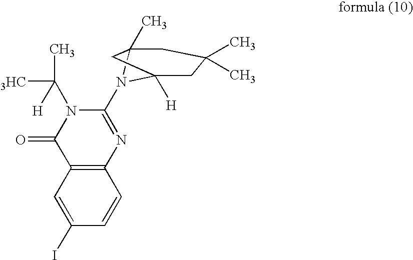

14. The compound according to claim 5 according to the following formula (11)

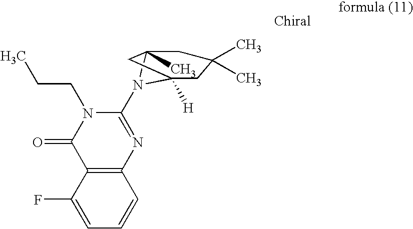

15. The compound according to claim 5 according to the following formula (12)

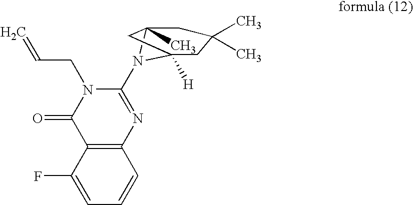

16. The compound according to claim 1 wherein said compound is capable of binding the NR1H3 receptor protein or a portion thereof according to SEQ ID NO. 1 or SEQ ID NO. 2 or a mammalian homologue thereof.

17. The compound according to claim 1 wherein said compound is capable of binding the NR1H2 receptor protein or a portion thereof or a mammalian homologue thereof.

18. A method for prevention or treatment of a NR1H3 and/or NR1H2 receptor protein mediated disease or NR1H3 and/or NR1H2 receptor protein homologue mediated disease or condition in a mammal comprising administering a therapeutically effective amount of a compound of the formula (1), or pharmaceutical acceptable salts or solvates thereof according to formula (1) 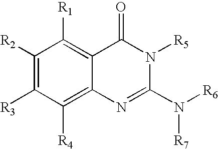
wherein:
R₁, R₂, R₃ and/or R₄, is independently from each other H, halogen, hydroxy, protected hydroxy, cyano, nitro, C₁ to C₆ alkyl, C₁ to C₆ substituted alkyl, C₁ to C₇ alkoxy, C₁ to C₇ substituted alkoxy, C₁ to C₇ acyl, C₁ to C₇ substituted acyl, C₁ to C₇ acyloxy, carboxy, protected carboxy, carboxymethyl, protected carboxymethyl, hydroxymethyl, protected hydroxymethyl, amino, protected amino, (monosubstituted)amino, protected (monosubstituted)amino, (disubstituted)amino, carboxamide, protected carboxamide, N—(C₁ to C₆ alkyl)carboxamide, protected N—(C₁ to C₆ alkyl)carboxamide, N, N-di(C₁ to C₆ alkyl)carboxamide, trifluoromethyl, N—((C₁ to C₆ alkyl)sulfonyl)amino, N-(phenylsulfonyl)amino or phenyl, wherein the phenyl is substituted or unsubstituted,
R₅ is H, C₁ to C₈ alkyl, C₁ to C₈ substituted alkyl, C₇ to C₁₂ alkylphenyl or C₇ to C₁₂ substituted phenylalkyl,
R₆ is H, C₁ to C₈ alkyl, C₁ to C₈ substituted alkyl, C₇ to C₁₂ alkylphenyl or C₇ to C₁₂ substituted phenylalkyl, and
R₇ is H, C₁ to C₈ alkyl, C₁ to C₈ substituted alkyl, C₇ to C₁₂ alkylphenyl or C₇ to C₁₂ substituted phenylalkyl; and
wherein the prevention or treatment is directly or indirectly accomplished through the binding of said compound to the NR1H3 and/or NR1H2 receptor proteins or to the NR1H3 and/or NR1H2 receptor protein homologues.

19. The method for prevention or treatment of a NR1H3 receptor protein and/or NR1H2 receptor protein mediated disease or condition according to claim 18, wherein said mammal is a human.

20. A method for:
i. regulating the cholesterol synthesis and/or transport in a mammal;
ii. treating, in a mammal, a disease which is affected by cholesterol, triglyceride, or bile acid levels;
iii. treating atherosclerosis, alzheimers disease, lipid disorders, obesity or a cardiovascular disorder in a mammal;
iv. blocking the cholesterol or fatty acid absorption in the intestine of a mammal;
v. blocking or treating obesity in a mammal; and/or
vi. modulating a gene whose expression is regulated by the NR1H3 and/or NR1H2 receptor in a mammal;
wherein said method comprises administering, to a mammal in need, a compound of the formula (1), or pharmaceutical acceptable salts or solvates thereof according to formula (1) 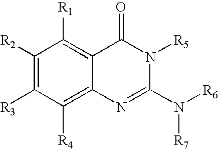
wherein:
R₁, R₂, R₃ and/or R₄, is independently from each other H, halogen, hydroxy, protected hydroxy, cyano, nitro, C₁ to C₆ alkyl, C₁ to C₆ substituted alkyl, C₁ to C₇ alkoxy, C₁ to C₇ substituted alkoxy, C₁ to C₇ acyl, C₁ to C₇ substituted acyl, C₁ to C₇ acyloxy, carboxy, protected carboxy, carboxymethyl, protected carboxymethyl, hydroxymethyl, protected hydroxymethyl, amino, protected amino, (monosubstituted)amino, protected (monosubstituted)amino, (disubstituted)amino, carboxamide, protected carboxamide, N—(C₁ to C₆ alkyl)carboxamide, protected N—(C₁ to C₆ alkyl)carboxamide, N, N-di(C₁ to C₆ alkyl)carboxamide, trifluoromethyl, N—((C₁ to C₆ alkyl)sulfonyl)amino, N-(phenylsulfonyl)amino or phenyl, wherein the phenyl is substituted or unsubstituted,
R₅ is H, C₁ to C₈ alkyl, C₁ to C₈ substituted alkyl, C₇ to C₁₂ alkylphenyl or C₇ to C₁₂ substituted phenylalkyl,
R₆ is H, C₁ to C₈ alkyl, C₁ to C₈ substituted alkyl, C₇ to C₁₂ alkylphenyl or C₇ to C₁₂ substituted phenylalkyl, and
R₇ is H, C₁ to C₈ alkyl, C₁ to C₈ substituted alkyl, C₇ to C₁₂ alkylphenyl or C₇ to C₁₂ substituted phenylalkyl.

21. The method according to claim 18 wherein the expression of ABCA1 and/or ABCG1 and/or ABCG5 and/or ABCG8 is increased.

22. The method according to claim 18 wherein the expression of the cholesterol 7 α hydroxylase and/or the activity of the cholesteryl ester transfer protein is increased.

23. The method according to claim 18 wherein the expression of the cholesterol 7 α hydroxylase and/or the activity of the cholesteryl ester transfer protein is enhanced.

24. A method for the selective up-regulation of one or more genes selected from the group consisting of ABCA1, ABCG1, ABCG5 and ABCG8 and a down-regulation of one or more of the genes selected from the group consisting of FAS and SREBP-1c, wherein the method comprises administering a compound of the formula (1), or pharmaceutical acceptable salts or solvates thereof according to formula (1) 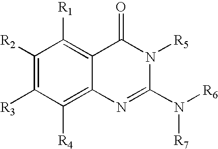
wherein:
R₁, R₂, R₃ and/or R₄, is independently from each other H, halogen, hydroxy, protected hydroxy, cyano, nitro, C₁ to C₆ alkyl, C₁ to C₆ substituted alkyl, C₁ to C₇ alkoxy, C₁ to C₇ substituted alkoxy, C₁ to C₇ acyl, C₁ to C₇ substituted acyl, C₁ to C₇ acyloxy, carboxy, protected carboxy, carboxymethyl, protected carboxymethyl, hydroxymethyl, protected hydroxymethyl, amino, protected amino, (monosubstituted)amino, protected (monosubstituted)amino, (disubstituted)amino, carboxamide, protected carboxamide, N—(C₁ to C₆ alkyl)carboxamide, protected N—(C₁ to C₆ alkyl)carboxamide, N, N-di(C₁ to C₆ alkyl)carboxamide, trifluoromethyl, N—((C₁ to C₆ alkyl)sulfonyl)amino, N-(phenylsulfonyl)amino or phenyl, wherein the phenyl is substituted or unsubstituted,
R₅ is H, C₁ to C₈ alkyl, C₁ to C₈ substituted alkyl, C₇ to C₁₂ alkylphenyl or C₇ to C₁₂ substituted phenylalkyl,
R₆ is H, C₁ to C₈ alkyl, C₁ to C₈ substituted alkyl, C₇ to C₁₂ alkylphenyl or C₇ to C₁₂ substituted phenylalkyl, and
R₇ is H, C₁ to C₈ alkyl, C₁ to C₈ substituted alkyl, C₇ to C₁₂ alkylphenyl or C₇ to C₁₂ substituted phenylalkyl; and
wherein said compound shows a larger difference in regulation of the two groups of genes when compared with the regulatory behavior of T0901317 on both groups of genes.

25. A pharmaceutical composition comprising a compound of the formula (1), or pharmaceutical acceptable salts or solvates thereof according to formula (1) 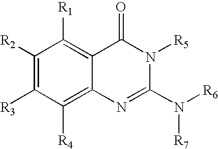
wherein:
R₁, R₂, R₃ and/or R₄, is independently from each other H, halogen, hydroxy, protected hydroxy, cyano, nitro, C₁ to C₆ alkyl, C₁ to C₆ substituted alkyl, C₁ to C₇ alkoxy, C₁ to C₇ substituted alkoxy, C₁ to C₇ acyl, C₁ to C₇ substituted acyl, C₁ to C₇ acyloxy, carboxy, protected carboxy, carboxymethyl, protected carboxymethyl, hydroxymethyl, protected hydroxymethyl, amino, protected amino, (monosubstituted)amino, protected (monosubstituted)amino, (disubstituted)amino, carboxamide, protected carboxamide, N—(C₁ to C₆ alkyl)carboxamide, protected N—(C₁ to C₆ alkyl)carboxamide, N, N-di(C₁ to C₆ alkyl)carboxamide, trifluoromethyl, N—((C₁ to C₆ alkyl)sulfonyl)amino, N-(phenylsulfonyl)amino or phenyl, wherein the phenyl is substituted or unsubstituted,
R₅ is H, C₁ to C₈ alkyl, C₁ to C₈ substituted alkyl, C₇ to C₁₂ alkylphenyl or C₇ to C₁₂ substituted phenylalkyl,
R₅ is H, C₁ to C₈ alkyl, C₁ to C₈ substituted alkyl, C₇ to C₁₂ alkylphenyl or C₇ to C₁₂ substituted phenylalkyl, and
R₇ is H, C₁ to C₈ alkyl, C₁ to C₈ substituted alkyl, C₇ to C₁₂ alkylphenyl or C₇ to C₁₂ substituted phenylalkyl;
wherein said compound is combined with a pharmaceutical carrier.
